(19) United States
(12) Patent Application Publication
(10) Pub. No.: US 2025/0130633 A1
(43) Pub. Date: Apr. 24, 2025

(54) INFORMATION PROCESSING APPARATUS, INFORMATION PROCESSING METHOD, AND PROGRAM

(71) Applicant: SONY GROUP CORPORATION, TOKYO (JP)

(72) Inventors: SHINICHI KAWANO, TOKYO (JP); NAOKI INOUE, TOKYO (JP); YUKI KAWANO, TOKYO (JP); HIRO IWASE, TOKYO (JP); TAKAYOSHI YAMASAKI, TOKYO (JP)

(21) Appl. No.: 18/694,380

(22) PCT Filed: Sep. 8, 2022
(86) PCT No.: PCT/JP2022/033648

§ 371 Date: Mar. 21, 2024

(30) Foreign Application Priority Data
Oct. 4, 2021 (JP) 2021-163658

(51) Int. Cl. G06F 3/01; G06T 11/60; G10L 15/26; G10L 25/63

(52) U.S. Cl. CPC G06F 3/013; G06T 11/60; G10L 15/26; G10L 25/63

(57) ABSTRACT

An information processing apparatus according to an embodiment of the present technology includes a first acquisition section, a second acquisition section, a display controller, and an estimator. The first acquisition section acquires text information obtained by converting speech of a speaker into a text using sound recognition. The second acquisition section acquires line-of-sight information that indicates a line of sight of a receiver who receives the speech of the speaker. The display controller displays the text information on at least a display apparatus that is used by the receiver. The estimator estimates, on the basis of the line-of-sight information, a viewing state of the receiver with respect to the text information displayed on the display apparatus used by the receiver. Further, the display controller controls display related to the text information on the basis of the viewing state.

### TECHNICAL FIELD

The present technology relates to an information processing apparatus, an information processing method, and a program that can be applied to, for example, a tool used for communication using sound recognition.

### BACKGROUND ART

Technologies that display contents of speech in the form of a text using sound recognition to assist communication have been developed in the past. Patent Literature 1 discloses a meeting system that converts a voice of a participant into a text and causes the text to be shared. In the system, a participant in a meeting is set to be a voice speaker or a text speaker. The voice of a voice speaker is reproduced for each participant. Further, the voice of a text speaker is converted into a text, and the text is displayed to the participants in a state of the text being combined with data of an image of the text speaker. Furthermore, the number of text speakers is limited when there are a large number of participants. This makes it possible to present speech in the form of a text clearly (for example, paragraphs [0052], [0121], and [0142] of the specification, and FIG. 7 in Patent Literature 1).

### CITATION LIST

### Patent Literature

Patent Literature 1: Japanese Patent Application Laid-open No. 2016-136746

### DISCLOSURE OF INVENTION

### Technical Problem

When contents of speech of a speaker are displayed in the form of a text, as described above, the concentration on the displayed text may result in difficulty in seeing, for example, an expression of a speaker, or conversely the concentration on, for example, the expression of the speaker may result in difficulty in checking the text. Thus, there is a need for a technology that makes it easy to check both what a speaker is like and contents of speech of the speaker.

In view of the circumstances described above, it is an object of the present technology to provide an information processing apparatus, an information processing method, and a program that make it possible to perform communication that enables a receiver to easily check both what a speaker is like and contents of speech of the speaker.

### Solution to Problem

In order to achieve the object described above, an information processing apparatus according to an embodiment of the present technology includes a first acquisition section, a second acquisition section, a display controller, and an estimator.

The first acquisition section acquires text information obtained by converting speech of a speaker into a text using sound recognition.

The second acquisition section acquires line-of-sight information that indicates a line of sight of a receiver who receives the speech of the speaker.

The display controller displays the text information on at least a display apparatus that is used by the receiver.

The estimator estimates, on the basis of the line-of-sight information, a viewing state of the receiver with respect to the text information displayed on the display apparatus used by the receiver.

Further, the display controller controls display related to the text information on the basis of the viewing state.

In the information processing apparatus, speech of the speaker is converted into a text using sound recognition, and the text is displayed on the display apparatus used by the receiver. Further, the viewing state of the receiver with respect to the text information is estimated on the basis of the line-of-sight information regarding the line of sight of the receiver. Display of the text information is controlled according to the viewing state. This makes it possible to display necessary text information when, for example, the receiver has not viewed the text information completely. This makes it possible to perform communication that enables the receiver to easily check both what the speaker is like and contents of speech of the speaker.

The estimator may perform a determination process of determining whether the viewing state of the receiver corresponds to a state in which the receiver has viewed the text information. In this case, the display controller may control the display related to the text information on the basis of a determination result obtained by the determination process related to the viewing state.

The estimator may perform the determination process related to the viewing state on the basis of at least one of the number of times of a back-and-forth movement of the line of sight of the receiver between a face of the speaker and the text information, a duration of the back-and-forth movement, or a remaining period of time for which the line of sight of the receiver remains on the face of the speaker.

The display controller may display the text information on the display apparatus used by the receiver in a state in which the text information is moving. In this case, the estimator may perform the determination process related to the viewing state on the basis of a following period of time for which the line of sight of the receiver follows the moving text information.

According to a speed at which the text information is updated, the estimator may change a determination threshold used to perform the determination process related to the viewing state.

When the receiver is in a state of not having viewed the text information, the display controller may set, as check-needed information, the text information not having been viewed by the receiver, and may cause the check-needed information to remain displayed on the display apparatus used by the receiver.

The display controller may display the check-needed information in a display mode that is different from a display mode used for other text information that is displayed on the same display screen as the check-needed information.

The display controller may determine a string of words, from among strings of words included in the check-needed information, that has been read by the receiver, and may display, in different display modes, the string of words having been read by the receiver and a string of words that has not been read by the receiver.

The display controller may display the check-needed information on the display apparatus used by the receiver, such that the check-needed information is stacked above the newly displayed text information.

The display apparatus used by the receiver may be a transmissive display apparatus. In this case, the display controller may display the check-needed information such that the check-needed information overlaps the face of the speaker.

The display apparatus used by the receiver may be a transmissive display apparatus. In this case, the display controller may display the check-needed information on a background viewed through the displayed apparatus used by the receiver, such that the check-needed information overlaps a portion of the background that makes the display of the check-needed information noticeable.

The information processing apparatus may further include an unnecessary information determining section that determines unnecessary information from among the check-needed information displayed on the display apparatus used by the receiver. In this case, the display controller may delete the display of the check-needed information determined to be the unnecessary information.

On the basis of the line-of-sight information regarding the line of sight of the receiver, the unnecessary information determining section may determine whether the receiver has checked the check-needed information, and the unnecessary information determining section may determine, as the unnecessary information, the check-needed information determined to have been checked by the receiver.

According to how frequently the receiver looks at the face of the speaker, the unnecessary information determining section may change a determination threshold used to perform a process of the determination related to the unnecessary information.

The unnecessary information determining section may determine, as the piece of unnecessary information, at least one of the piece of check-needed information displayed on the display apparatus used by the receiver for a period of time that exceeds a threshold, or the piece of check-needed information displayed on the display apparatus used by the receiver for a longest period of time at a timing at which the number of the pieces of check-needed information displayed on the display apparatus used by the receiver exceeds a threshold.

The information processing apparatus may further include a feeling estimator that estimates feeling information that indicates a feeling of the speaker during speaking. When the receiver is in a state of having viewed the text information, the display controller may perform an arrangement process of arranging the text information according to the feeling information regarding the feeling of the speaker.

The display controller may decorate the text information according to the feeling information regarding the feeling of the speaker, or may add a visual effect to a portion situated around the text information, according to the feeling information regarding the feeling of the speaker.

When the receiver is in a state of not having viewed the text information, the display controller may generate a report image used to inform that the receiver has not viewed the text information, and displays the generated report image on a display apparatus that is used by the speaker.

An information processing method according to an embodiment of the present technology is an information processing method that is performed by a computer system, the information processing method including acquiring text information obtained by converting speech of a speaker into a text using sound recognition.

Line-of-sight information that indicates a line of sight of a receiver who receives the speech of the speaker is acquired.

The text information is displayed on at least a display apparatus that is used by the receiver.

A viewing state of the receiver with respect to the text information displayed on the display apparatus used by the receiver is estimated on the basis of the line-of-sight information.

Display related to the text information is controlled on the basis of the viewing state.

A program according to an embodiment of the present technology causes a computer system to perform a process including:
acquiring text information obtained by converting speech of a speaker into a text using sound recognition;
acquiring line-of-sight information that indicates a line of sight of a receiver who receives the speech of the speaker;
displaying the text information on at least a display apparatus that is used by the receiver;
estimating, on the basis of the line-of-sight information, a viewing state of the receiver with respect to the text information displayed on the display apparatus used by the receiver; and
controlling display related to the text information on the basis of the viewing state.

### BRIEF DESCRIPTION OF DRAWINGS

FIG. 1 schematically illustrates an overview of a communication system according to an embodiment of the present technology.

FIG. 2 schematically illustrates examples of display screens that are respectively viewed by a speaker and a receiver.

FIG. 3 is a block diagram illustrating an example of a configuration of the communication system.

FIG. 4 is a block diagram illustrating an example of a configuration of a system controller.

FIG. 5 is a flowchart illustrating an example of an operation of the communication system on a receiver side.

FIG. 6 schematically illustrates examples of presenting text information to the receiver.

FIG. 7 is a flowchart illustrating an example of a determination process related to a viewing state of the receiver.

FIG. 8 is a schematic diagram used to describe the determination process illustrated in FIG. 7.

FIG. 9 is a flowchart illustrating another example of the determination process related to the viewing state of the receiver.

FIG. 10 is a schematic diagram used to describe the determination process illustrated in FIG. 9.

FIG. 11 is a flowchart illustrating another example of the determination process related to the viewing state of the receiver.

FIG. 12 is a schematic diagram used to describe the determination process illustrated in FIG. 11.

FIG. 13 schematically illustrates examples of a process of presenting check-needed information to the receiver.

FIG. 14 schematically illustrates examples of the process of presenting check-needed information to the receiver.

FIG. 15 schematically illustrates an example of a process of deleting display of the check-needed information.

FIG. 16 schematically illustrates examples of a process of decorating text information in a state of having been viewed by the receiver.

### MODE(S) FOR CARRYING OUT THE INVENTION

Embodiments according to the present technology will now be described below with reference to the drawings.

### [Configuration of Communication System]

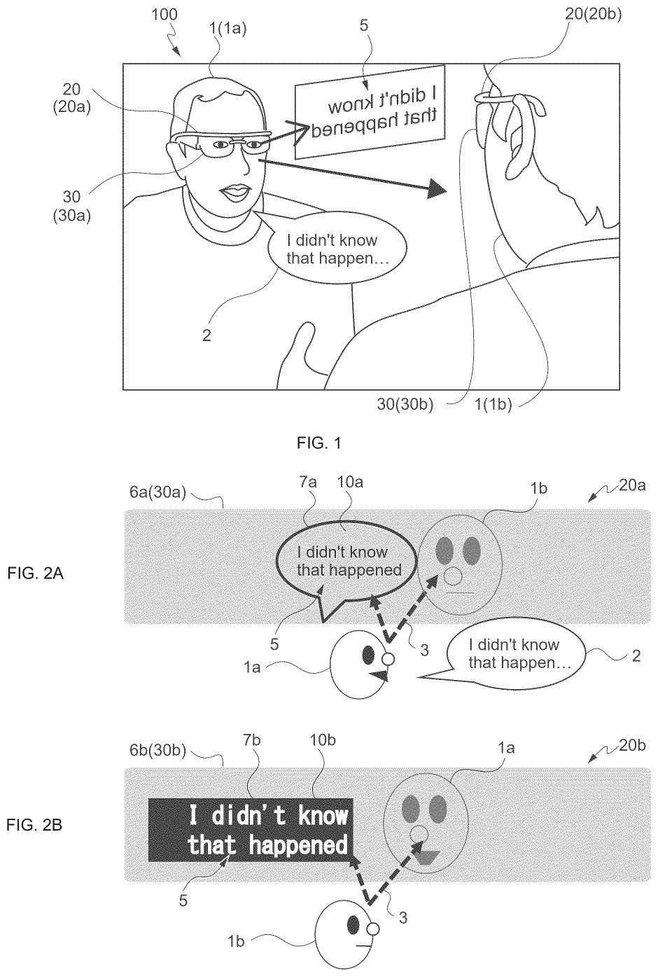

FIG. 1 schematically illustrates an overview of a communication system according to an embodiment of the present technology. A communication system 100 is a system that assists communication between users 1 by displaying text information 5 that is obtained using sound recognition. The communication system 100 is used when, for example, the hearing ability is restricted.

Examples of the case in which the hearing ability is restricted include having a conversation in a loud environment, having a conversation in different languages, and the case in which the user 1 is a deaf or hard-of-hearing person. In such cases, the use of the communication system 100 makes it possible to have a conversation using text information 5.

In the communication system 100, smart glasses 20 are used as an apparatus used to display the text information 5. The smart glasses 20 are an eyeglass-type head mounted display (HMD) terminal that includes a transmissive display 30.

The user 1 who is wearing the smart glasses 20 views the outside world through the transmissive display 30. Here, various visual information including the text information 5 is displayed on the display 30. This enables the user 1 to view the visual information superimposed on the real world and to check the text information 5 during communication.

In the present embodiment, the smart glasses 20 are an example of a transmissive display apparatus.

FIG. 1 schematically illustrates two users 1a and 1b communicating with each other using the communication system 100. The users 1a and 1b are respectively wearing smart glasses 20a and smart glasses 20b.

In FIG. 1, sound recognition is performed on a voice 2 of the user 1a, and the text information 5 obtained by converting contents of speech of the user 1a into a text is generated. The text information 5 is displayed on both the smart glasses 20a used by the user 1a and the smart glasses 20b used by the user 1b. This results in the user 1a and the user 1b communicating each other using the text information 5.

In the following description, it is assumed that the user 1a is a person with good hearing and the user 1b is a deaf or hard-of-hearing person. Further, the user 1a is referred to as a speaker 1a and the user 1b is referred to as a receiver 1b.

FIG. 2 schematically illustrates examples of display screens that are respectively viewed by the speaker 1a and the receiver 1b. A of FIG. 2 schematically illustrates a display screen 6a that is displayed on a display 30a of the smart glasses 20a worn by the speaker 1a. Further, B of FIG. 2 schematically illustrates a display screen 6b displayed on a display 30b of the smart glasses 20b worn by the receiver 1b.

Further, A and B of FIG. 2 illustrate changes in respective lines of sight 3 (dotted arrows) of the speaker 1a and the receiver 1b. The speaker 1a (the receiver 1b) moves his/her own line of sight 3, and this enables the speaker 1a (the receiver 1b) to view various information displayed on the display screen 6a (the display screen 6b) and the outside world seen through the display screen 6a (the display screen 6b).

In the communication system 100, sound recognition is performed on the voice 2 of the speaker 1a, and a string of words (the text information 5) that indicates contents of speech of the voice 2 is generated. Here, the speaker 1a speaks “I didn't know that happened”, and a string of words that indicates “I didn't know that happened” is generated as the text information 5. The text information 5 is displayed on each of the display screens 6a and 6b in real time. Note that the displayed text information 5 is a string of words that is an intermediate result of sound recognition or a final determined result of sound recognition. Further, the text information 5 is not necessarily have to correspond to contents of speech of the speaker 1, and a wrong string of words may be displayed.

The text information 5 obtained using sound recognition is displayed on the smart glasses 20a with no change, as illustrated in A of FIG. 2. In other words, the string of words “I didn't know that happened” is displayed. In the example illustrated in A of FIG. 2, the text information 5 is displayed inside of an object 7a in the form of a balloon. In this case, a region inside of the object 7a is a text display region 10a in which the text information 5 presented to the speaker 1a is displayed.

Further, the speaker 1a can view the receiver 1b through the display screen 6a. Basically, the object 7a including the text information 5 is displayed not to overlap the receiver 1b.

As described above, the text information 5 is presented to the speaker 1a, and this enables the speaker 1a to check the text information 5 obtained by converting contents of his/her own speech into a text. Thus, if, for example, the text information 5 different from the text information 5 corresponding to contents of speech of the speaker 1a is displayed due to sound recognition being erroneously performed, the speaker 1a can speak once more or inform the receiver 1b that the text information 5 is wrong.

Further, the speaker 1a can check a face of the receiver 1b through the display screen 6a (the display 30a). This enables natural communication.

Likewise, the text information 5 obtained using sound recognition is displayed on the smart glasses 20b with no change, as illustrated in B of FIG. 2. In other words, the string of words “I didn't know that happened” is displayed. In the example illustrated in B of FIG. 2, the text information 5 is displayed inside of a rectangular object 7b. Here, the text information 5 is displayed as a string of words in white in a black background. In this case, a region inside of the object 7b is a text display region 10b in which the text information 5 presented to the receiver 1b is displayed.

Further, the receiver 1b can view the speaker 1a through the display screen 6b. Basically, the object 7b including the text information 5 is displayed not to overlap the speaker 1a.

As described above, the text information 5 is presented to the receiver 1b, and this enables the receiver 1b to check contents of speech of the speaker 1a in the form of the text information 5. This enables communication using the text information 5 if the receiver 1b does not catch the voice 2.

Further, the receiver 1b can check a face of the speaker 1a through the display screen 6b (the display 30b). This enables the receiver 1b to easily check information, such as a movement of lips of and an expression of the speaker 1a, that is other than the text information.

For example, when a process only including converting contents of speech into a text using sound recognition and displaying text information corresponding to the text is performed, elements (nonverbal), such as the expression and gestures in a conversation, that are other than language are not converted into a text. On the other hand, nonverbal information is a very important element in order to grasp a shade of meaning in conversation and counterpart's feeling. Thus, when the receiver 1b who is a deaf or hard-of-hearing person receives speech of the speaker 1a, the expression and gestures of the speaker 1a are important for the receiver 1b in order to acquire nonverbal information.

In the communication system 100, the receiver 1b reads nonverbal information regarding the speaker 1a from the face and the body of the speaker 1a seen through the display 30b.

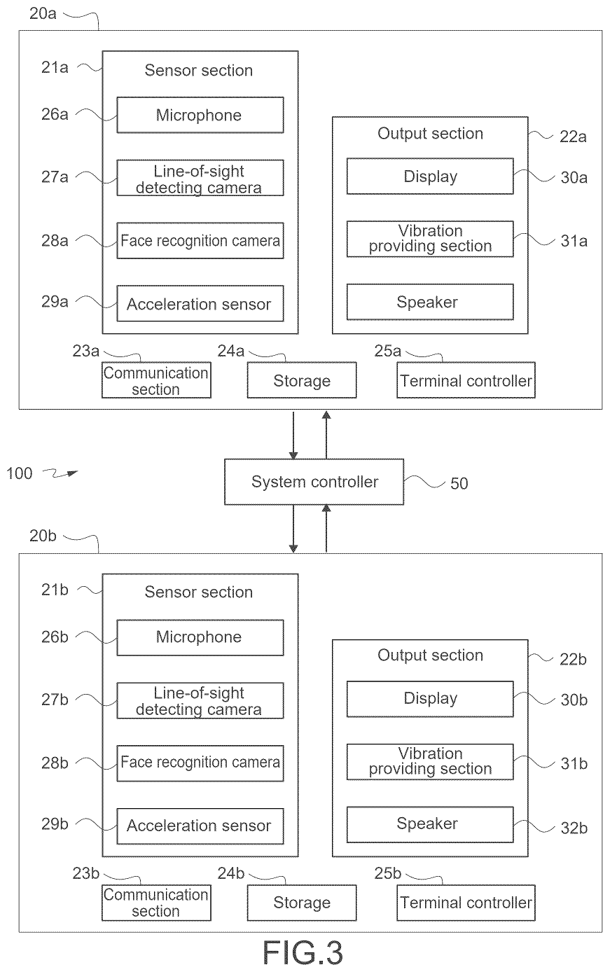

FIG. 3 is a block diagram illustrating an example of a configuration of the communication system 100. As illustrated in FIG. 3, the communication system 100 includes the smart glasses 20a, the smart glasses 20b, and a system controller 50.

Here, the smart glasses 20a and the smart glasses 20b have similar configurations, where a structural element for the smart glasses 20a is denoted by reference numeral with “a” and a structural element for the smart glasses 20b is denoted by reference numeral with “b”.

First, the configuration of the smart glasses 20a is described. The smart glasses 20a are an eyeglass-type display apparatus, and include a sensor section 21a, an output section 22a, a communication section 23a, a storage 24a, and a terminal controller 25a.

The sensor section 21a includes, for example, a plurality of sensor elements provided to a housing of the smart glasses 20a, and includes a microphone 26a, a line-of-sight detecting camera 27a, a face recognition camera 28a, and an acceleration sensor 29a.

The microphone 26a is a sound collection element that collects the voice 2, and is provided to the housing of the smart glasses 20a such that the voice 2 of a wearing person (here, the speaker 1a) can be collected.

The line-of-sight detecting camera 27a is an inward-oriented camera used to capture an image of eyeballs of the wearing person. An image of the eyeballs that is captured using the line-of-sight detecting camera 27a is used to detect a line of sight 3 of the wearing person. The line-of-sight detecting camera 27a is, for example, a digital camera that includes an image sensor such as a complementary metal-oxide semiconductor (CMOS) sensor or a charge-coupled device (CCD) sensor. Further, the line-of-sight detecting camera 27a may be an infrared camera. In this case, an infrared light source or the like that irradiates infrared light onto the eyeballs of the wearing person may be provided. Such a configuration makes it possible to detect a line of sight with a high degree of accuracy on the basis of an infrared image of the eyeballs.

The face recognition camera 28a is an outward-oriented camera used to perform image-capturing in a range that is similar to the range of a field of view of the wearing person. An image captured using the face recognition camera 28a is used to, for example, detect a face of a communication counterpart (here, the receiver 1b) of the wearing person. The face recognition camera 28a is a digital camera that includes an image sensor such as a CMOS sensor or a CCD sensor.

The acceleration sensor 29a is a sensor that detects acceleration of the smart glasses 20a. Output of the acceleration sensor 29a is used to, for example, detect an orientation (a pose) of a head of the wearing person. For example, a nine-axis sensor that includes a three-axis acceleration sensor, a three-axis gyroscope, and a three-axis compass sensor is used as the acceleration sensor 29a.

The output section 22a includes a plurality of output elements providing information and a stimulus to the wearing person who is wearing the smart glasses 20a, and includes the display 30a, a vibration providing section 31a, and a speaker 32a.

The display 30a is a transmissive display element, and is fixed to the housing of the smart glasses 20a to be situated in front of the eyes of the wearing person. The display 30a is formed using a display element such as a liquid crystal display (LCD) or an organic EL display. The smart glasses 20a include a left-eye display and a right-eye display. The left-eye display and the right-eye display respectively display, to the left eye and the right eye of the wearing person, images corresponding to the respective eyes.

Alternatively, a configuration in which a single display is provided and the same image is displayed to the two eyes of the wearing person, or a configuration in which an image is displayed to only one of the left eye and the right eye of the wearing person may be used.

The vibration providing section 31a is a vibrational element that provides vibration to the wearing person. An element, such as an eccentric motor or a voice coil motor (VCM), that can generate vibration is used as the vibration providing section 31a. For example, the vibration providing section 31a is provided in the housing of the smart glasses 20a. Note that a vibrational element that is provided to another apparatus (such as a mobile terminal or a wearable terminal) that is used by the wearing person may be used as the vibration providing section 31a.

The speaker 32a is a sound reproduction element that reproduces sound so that the wearing person can hear the sound. For example, the speaker 32a is included in the housing of the smart glasses 20a as a built-in speaker. Further, the speaker 32a may be earphones or a headphone that is used by the wearing person.

The communication section 23a is a module used to perform, for example, network communication or near field communication with another device. For example, a wireless LAN module such as Wi-Fi, or a communication module such as Bluetooth (registered trademark) is provided as the communication section 23a. Moreover, for example, a communication module that enables communication using wired connection may be provided.

The storage 24a is a nonvolatile storage device. For example, a recording medium using a solid-state device such as a solid-state drive (SSD), or a magnetic recording medium such as a hard disk drive (HDD) is used as the storage 24a. Moreover, a type and the like of a recording medium used as the storage 24a are not limited, and, for example, any recording medium that non-transiently records therein data may be used. The storage 24a stores therein, for example, a program that controls an operation of each structural element of the smart glasses 20a.

The terminal controller 25a controls an operation of the smart glasses 20a. The terminal controller 25a is configured by hardware, such as a CPU and a memory (a RAM and a ROM), that is necessary for a computer. Various processes are performed by the CPU loading, into the RAM, the program stored in the storage 24a and executing the program.

Next, the configuration of the smart glasses 20b is described. The smart glasses 20b are an eyeglass-type display apparatus, and include a sensor section 21b, an output section 22b, a communication section 23b, a storage 24b, and a terminal controller 25b. Further, the sensor section 21b includes a microphone 26b, a line-of-sight detecting camera 27b, a face recognition camera 28b, and an acceleration sensor 29b. Furthermore, the output section 22b includes the display 30b, a vibration providing section 31b, and a speaker 32b.

For example, the structural elements of the smart glasses 20b are similar to the structural elements of the smart glasses 20a described above. Further, the description of the structural elements of the smart glasses 20a can also be used as the description of the structural elements of the smart glasses 20b, with the wearing person being replaced with the receiver 1b.

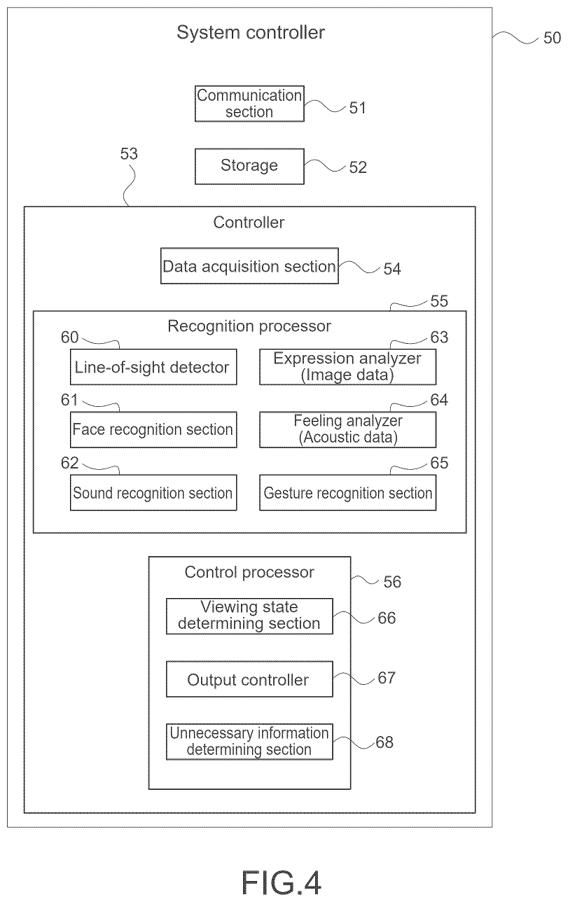

FIG. 4 is a block diagram illustrating an example of a configuration of the system controller 50. The system controller 50 is a control apparatus that controls an operation of the overall communication system 100, and includes a communication section 51, a storage 52, and a controller 53.

Here, it is assumed that the system controller 50 is a server apparatus that can communicate with the smart glasses 20a and the smart glasses 20b through a specified network. Note that the system controller 50 may be a terminal apparatus (for example, a smartphone or a tablet terminal) that can directly communicate with the smart glasses 20a and the smart glasses 20b without using, for example, a network.

The communication section 51 is a module used for, for example, network communication or near field communication performed between the system controller 50 and other devices such as the smart glasses 20a and the smart glasses 20b. For example, a wireless LAN module such as Wi-Fi, or a communication module such as Bluetooth (registered trademark) is provided as the communication section 51. Moreover, for example, a communication module that enables communication using wired connection may be provided.

The storage 52 is a nonvolatile storage device. For example, a recording medium using a solid-state device such as an SSD, or a magnetic recording medium such as an HDD is used as the storage 52. Moreover, a type and the like of a recording medium used as the storage 52 are not limited, and, for example, any recording medium that non-transiently records therein data may be used.

The storage 52 stores therein a control program according to the present embodiment. The control program is a program that controls an operation of the overall communication system 100. Further, the storage 52 stores therein, for example, a history of the text information 5 obtained using sound recognition, and a log that stores therein states (such as a change in the line of sight 3, a speed of speech, and the volume) of the speaker 1a and receiver 1b in communication with each other.

Moreover, information stored in the storage 52 is not limited.

The controller 53 controls an operation of the communication system 100. The controller 53 is configured by hardware, such as a CPU and a memory (a RAM and a ROM), that is necessary for a computer. Various processes are performed by the CPU loading, into the RAM, the control program stored in the storage 52 and executing the program. The controller 53 corresponds to an information processing apparatus according to the present embodiment.

For example, a programmable logic device (PLD) such as a field programmable gate array (FPGA), or another device such as an application specific integrated circuit (ASIC) may be used as the controller 53. Further, for example, a processor such as a graphics processing unit (GPU) may be used as the controller 53.

In the present embodiment, a data acquisition section 54, a recognition processor 55, and a control processor 56 are implemented as functional blocks by the CPU of the controller 53 executing a program (the control program) according to the present embodiment. Then, an information processing method according to the present embodiment is performed by these functional blocks. Note that, in order to implement each functional block, dedicated hardware such as an integrated circuit (IC) may be used as appropriate.

The data acquisition section 54 acquires data necessary for operations of the recognition processor 55 and the control processor 56 as appropriate. For example, the data acquisition section 54 reads, for example, sound data and image data from the smart glasses 20a and the smart glasses 20b through the communication section 51. Further, for example, data obtained by recording states of the speaker 1a and the receiver 1b that are stored in the storage 52 is read as appropriate.

The recognition processor 55 performs various recognition processes (such as face recognition, line-of-sight detection, sound recognition, expression analysis, feeling analysis, and gesture recognition) on the basis of data output from the smart glasses 20a and the smart glasses 20b. Here, recognition processes performed in order to control the smart glasses 20b used by the receiver 1b are primarily described.

As illustrated in FIG. 4, the recognition processor 55 includes a line-of-sight detector 60, a face recognition section 61, a sound recognition section 62, an expression analyzer 63, a feeling analyzer 64, and a gesture recognition section 65. A process performed by the line-of-sight detector 60 from among these structural elements is a recognition process (line-of-sight recognition) that is performed on the receiver 1b. On the other hand, processes performed by the face recognition section 61, the sound recognition section 62, the expression analyzer 63, the feeling analyzer 64, and the gesture recognition section 65 are recognition processes (face recognition, sound recognition, expression analysis, feeling analysis, and gesture recognition) that are performed on the speaker 1a.

The line-of-sight detector 60 detects the line of sight 3 of the receiver 1b. Specifically, the line-of-sight detector 60 acquires line-of-sight information that indicates the line of sight 3 of the receiver 1b. Here, the line-of-sight information is information that can indicate a line of sight of the receiver 1b. For example, the line of sight 3 of the receiver 1b is detected on the basis of data of an image of the eyeballs of the receiver 1b that is captured using the line-of-sight detecting camera 27b included in the smart glasses 20b. The process may calculate a vector that indicates an orientation of the line of sight 3, or may calculate a point of intersection (a viewpoint) of the display screen 6a and the line of sight 3. Information regarding a vector of the line of sight 3 and position information regarding a position of the viewpoint both correspond to the line-of-sight information.

A specific method for performing the line-of-sight detecting process is not limited. For example, the cornea-reflex method is used when, for example, an infrared camera is used as the line-of-sight detecting camera 27b. Further, for example, a method for detecting the line of sight 3 on the basis of a position of a pupil (an iris) may be used.

In the present embodiment, the line-of-sight detector 60 corresponds to a second acquisition section that acquires line-of-sight information.

The face recognition section 61 performs a face recognition process on data of an image captured using the face recognition camera 28b included in the smart glasses 20b. In other words, the face of the speaker 1a is detected in an image in a field of view of the receiver 1b. Further, from a result of the detection of the face of the speaker 1a, the face recognition section 61 estimates, for example, a position and a region of the face of the speaker 1a on the display screen 6b viewed by the receiver 1b (refer to B of FIG. 2). Moreover, the face recognition section 61 may estimate, for example, an orientation of the head of the speaker 1a.

A specific method for performing the face recognition process is not limited. For example, any face detection technologies using, for example, feature-amount detection or machine learning may be used.

The sound recognition section 62 performs a sound recognition process on the basis of sound data obtained by collecting the voice 2 of the speaker 1a. In this process, contents of speech of the speaker 1a are converted into a text and output in the form of the text information 5. As described above, the sound recognition section 62 acquires text information obtained by converting speech of the speaker 1a into a text using sound recognition. In the present embodiment, the sound recognition section 62 corresponds to a first acquisition section that acquires text information.

Sound data used to perform the sound recognition process is typically data of sound collected by the microphone 26a included in the smart glasses 20a worn by the speaker 1a. Note that data of sound collected by the microphone 26b of the receiver 1b may be used to perform the sound recognition process performed on the speaker 1a.

In the present embodiment, the sound recognition section 62 successively outputs the pieces of text information 5 estimated during the sound recognition process in addition to the piece of text information 5 calculated as a final result obtained by the sound recognition process. Thus, for example, the text information 5 corresponding to an intermediate syllable in the text information 5 corresponding to the final result is output before the text information 5 corresponding to the final result is displayed. Note that the text information 5 may be converted into, for example, kanji, katakana, or the alphabet as appropriate to be output.

Further, the sound recognition section 62 may calculate a degree of reliability of the sound recognition process (a degree of certainty of the text information 5) together with the text information 5.

A specific method for performing the sound recognition process is not limited. Any sound recognition technologies such as sound recognition using an acoustic model or a language model and sound recognition using machine learning may be used.

The expression analyzer 63 performs an expression analysis process of analyzing the expression of the speaker 1a. For example, this process is an image analysis process performed to estimate the expression of the speaker 1a from data of an image captured using the face recognition camera 28b included in the smart glasses 20b. For example, a degree of smiling and a type of feeling (such as delight and surprise) of the speaker 1a are estimated with respect to the face of the speaker 1a that is recognized by the face recognition section 61.

A specific method for performing the expression analysis process is not limited. For example, a method for classifying expressions on the basis of a positional relationship between feature points (such as a pupil, a corner of a mouth, and a nose) on a human face, or a method for estimating a feeling using, for example, machine learning may be used.

The feeling analyzer 64 performs a feeling analysis process of analyzing a feeling of the speaker 1a. This process is, for example, an acoustic analysis process performed to estimate the feeling of the speaker 1a from data of sound collected by the microphone 26a of the smart glasses 20a and by the microphone 26b of the smart glasses 20b. For example, the type of feeling of the speaker 1a when the speaker 1a produces the voice 2 is estimated for the voice 2.

A specific method for performing the feeling analysis process is not limited. For example, it is known that the rhythm of voice is changed according to the feeling. Such a change in rhythm is extracted in the form of a feature-amount vector, and this makes it possible to estimate a feeling included in speech. Pattern recognition with respect to a feature-amount vector and a process of classifying feelings using, for example, machine learning are used in this process.

The gesture recognition section 65 performs a gesture recognition process of recognizing a gesture of the speaker 1a. This process is, for example, an image analysis process performed to estimate a gesture of the speaker 1a from data of an image captured using the face recognition camera 28b included in the smart glasses 20b.

In the present embodiment, a gesture that represents the feeling of the speaker 1a is recognized. For example, a gesture of the speaker 1a putting his/her hands on his/her head is a gesture that represents confusion. Further, a gesture of the speaker 1a raising his/her hands above the level of his/her face is a gesture that represents surprising. Moreover, any other gestures that represent feelings may be detected. Further, for example, a gesture that describes contents of speech (a gesture that represents, for example, a size, a speed, or a length) may be detected.

A specific method for performing the gesture recognition process is not limited. For example, a method for recognizing a gesture on the basis of a positional relationship between feature points (such as a palm of a hand, a finger, a shoulder, a breast, and a head) of a human body, or a method for recognizing a gesture using, for example, machine learning may be used.

The pieces of information estimated by the expression analyzer 63, the feeling analyzer 64, and the gesture recognition section 65 are examples of pieces of feeling information indicating feelings such as delight and surprise of the speaker 1a. These pieces of information are used to perform, for example, a process of decorating the text information 5 (refer to, for example, FIG. 16).

In the present embodiment, the expression analyzer 63, the feeling analyzer 64, and the gesture recognition section 65 serve as a feeling estimator that estimates feeling information that indicates the feeling of the speaker 1a during speaking.

The control processor 56 performs various processes in order to control operations of the smart glasses 20a and the smart glasses 20b.

As illustrated in FIG. 4, the control processor 56 includes a viewing state determining section 66, an output controller 67, and an unnecessary information determining section 68.

On the basis of line-of-sight information regarding the receiver 1b, the viewing state determining section 66 estimates a viewing state of the receiver 1b with respect to the text information 5 displayed on the smart glasses 20b used by the receiver 1b. In the present embodiment, the viewing state determining section 66 corresponds to an estimator that estimates a viewing state.

In the present disclosure, the viewing state corresponds to a state in which, for example, the receiver 1b looks at the text information 5 displayed on the smart glasses 20b and recognizes contents of the text information 5 (a state in which the receiver 1b views the text information 5). The viewing state includes, for example, two states that are a state in which the receiver 1b has viewed the text information 5, and a state in which the receiver 1b has not viewed the text information 5. Alternatively, the viewing state can also be represented by to what extent the receiver 1b has viewed the text information 5.

In the present embodiment, the viewing state determining section 66 performs a determination process related to a viewing state. The determination process related to a viewing state is a process of determining whether the viewing state of the receiver 1b corresponds to a state in which the receiver 1b has viewed the text information 5.

The viewing state determining section 66 reads the above-described line-of-sight information regarding the line of sight of the receiver 1b that is acquired by the line-of-sight detector 60, and performs the determination process related to a viewing state on the basis of the line-of-sight information.

For example, when a sufficient period of time has elapsed since the line of sight 3 of the receiver 1b started being oriented toward the text information 5, it is determined that the receiver 1b is in a state of having viewed the text information 5. In this case, it is considered that the receiver 1b has read the text information 5. On the other hand, for example, when the line of sight 3 of the receiver 1b is not oriented toward the text information 5, or when the line of sight 3 of the receiver 1b is oriented toward the text information 5 for only a short period of time, it is determined that the receiver 1b is in a state of not having viewed the text information 5. In this case, it is considered that the receiver 1b has not read the text information 5.

As described above, the determination process related to a viewing state can also be a process of determining whether the receiver 1b has read the text information 5 displayed on the smart glasses 20b.

The output controller 67 controls an operation of the output section 22a provided to the smart glasses 20a and an operation of the output section 22b provided to the smart glasses 20b.

Specifically, the output controller 67 generates data to be displayed on the display 30a (the display 30b). The generated data is output to the smart glasses 20a (the smart glasses 20b), and display performed on the display 30a (the display 30b) is controlled. This data includes, for example, data of the text information 5 and data that specifies, for example, a display position for the text information 5. In other words, it can also be said that the output controller 67 performs display control on the display 30a (the display 30b). As described above, in the present embodiment, the output controller 67 performs a process of displaying the text information 5 on the smart glasses 20a used by the speaker 1a and on the smart glasses 20b used by the receiver 1b. In the present embodiment, the output controller 67 serves as a display controller.

Further, the output controller 67 generates, for example, vibration data that specifies, for example, a vibration pattern of the vibration providing section 31a (the vibration providing section 31b), and data of sound reproduced by the speaker 32a (the speaker 32b). The vibration data and the sound data are used to control presentation of vibration on the smart glasses 20a (the smart glasses 20b) and reproduction of sound in the smart glasses 20a (the smart glasses 20b).

Further, the output controller 67 controls display related to the text information 5 on the basis of the viewing state of the receiver 1b. More specifically, display related to display of the text information 5 that is performed on the smart glasses 20b used by the receiver 1b and on the smart glasses 20a used by the speaker 1a is controlled on the basis of the viewing state of the receiver 1b. In this process, for example, contents of, a display form of, a display position for, movement of, and a decorative effect of the displayed text information 5 are control targets.

Control on display performed on the smart glasses 20b (the display screen 6b of the display 30b) used by the receiver 1b is primarily described below.

In the present embodiment, the output controller 67 controls display related to the text information 5 on the basis of a determination result obtained by the above-described determination process related to the viewing state of the receiver 1b that is performed by the viewing state determining section 66. In other words, display contents of the text information 5 to be viewed by the receiver 1b are selected and a display form of the text information 5 to be viewed by the receiver 1b is adjusted using the result of determination of whether the receiver 1b has viewed the text information 5.

For example, when the receiver 1b is in a state of not having viewed the text information 5, it is considered that the text information 5 displayed on the smart glasses 20b is the text information 5, which has not been read by the receiver 1b. The output controller 67 sets, to be check-needed information, unread text information 5 considered not to have been read by the receiver 1b, as described above, and causes the unread text information 5 to remain displayed on the smart glasses 20b.

Note that, from among the check-needed information remaining displayed on the smart glasses 20b, information that becomes unnecessary is deleted as appropriate. Consequently, display of check-needed information is not increased unnecessarily.

The unnecessary information determining section 68 determines unnecessary information from among check-needed information displayed on the smart glasses 20b used by the receiver 1b.

As described above, in the present embodiment, the output controller 67 displays check-needed information on the smart glasses 20b (the display screen 6b). The unnecessary information determining section 68 determines unnecessary information from among displayed check-needed information, as described above. Display of check-needed information determined to be unnecessary information is deleted by the output controller 67.

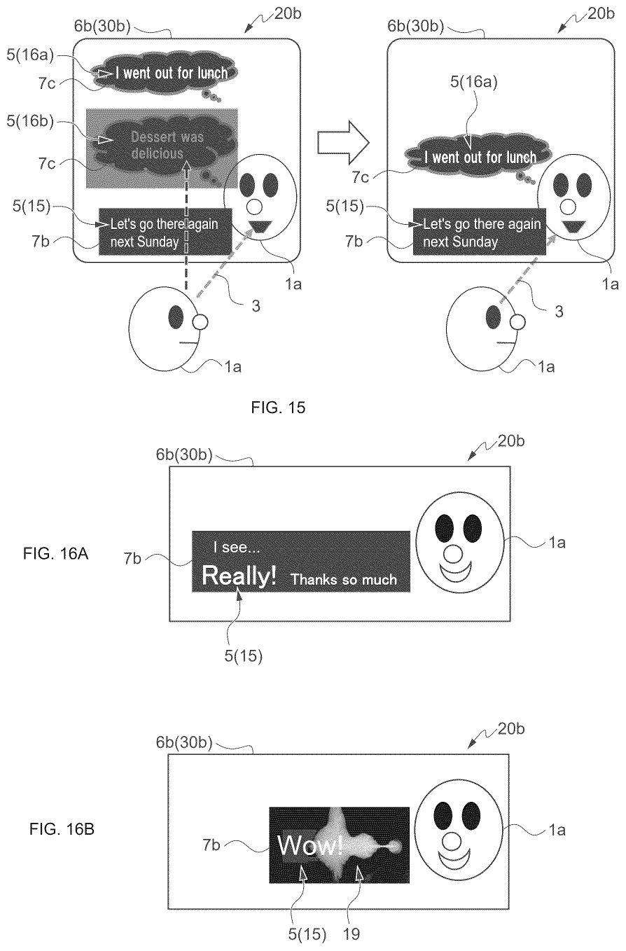

A specific method for determining unnecessary information will be described later in detail with reference to FIG. 15.

The example in which the system controller 50 is a server apparatus or a terminal apparatus has been described above. However, the configuration of the system controller 50 is not limited thereto.

For example, the smart glasses 20a (the smart glasses 20b) may be the system controller 50. In this case, the communication section 23a (the communication section 23b) serves as the communication section 51, the storage 24a (the storage 24b) serves as the storage 52, and the terminal controller 25a (the terminal controller 25b) serves as the controller 53. Further, the functions of the system controller 50 (the controller 53) may be provided separately from each other. For example, the functional blocks of the recognition processor 55 may be implemented by, for example, a server apparatus dedicated to recognition processes.

### [Operation of Communication System on Receiver Side]

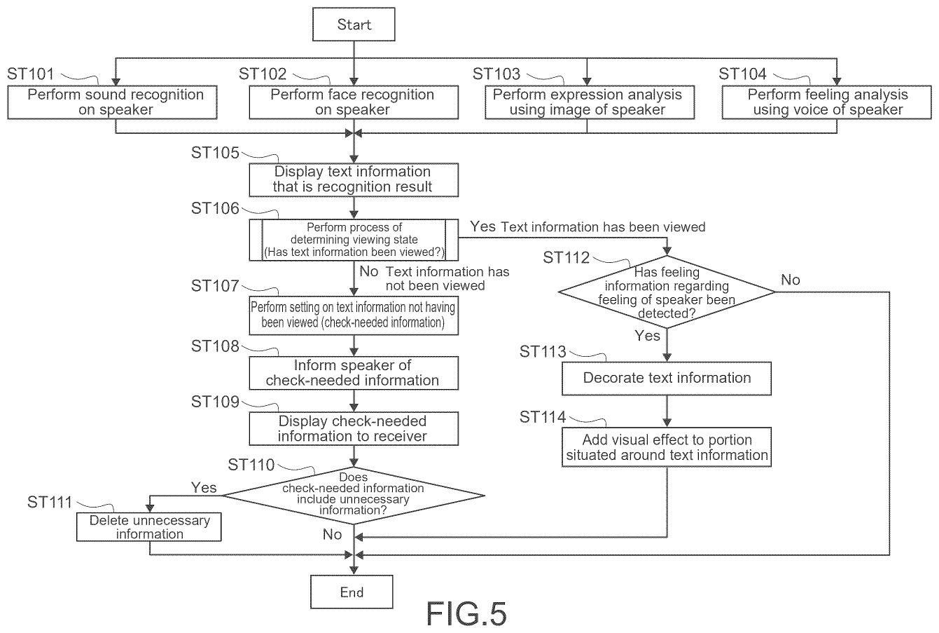

FIG. 5 is a flowchart illustrating an example of the operation of the communication system 100 on a side of the receiver 1b. The process illustrated in FIG. 5 is a process of primarily controlling an operation of the smart glasses 20b used by the receiver 1b, and is repeatedly performed during communication between the speaker 1a and the receiver 1b. The operation of the communication system 100 for the receiver 1b is described below with reference to FIG. 5.

First, various recognition processes related to the speaker 1a are performed in parallel (Steps 101 to 104). For example, these processes may be continuously performed in a background, or may be performed in response to speech of the speaker 1a being detected.

In Step 101, the sound recognition section 62 performs sound recognition with respect to the voice 2 of the speaker 1a. For example, the voice 2 of the speaker 1a is collected by the microphone 26a of the smart glasses 20a. The collected data is input to the sound recognition section 62 of the system controller 50. The sound recognition section 62 performs the sound recognition process with respect to the voice 2 of the speaker 1a, and outputs the text information 5. The text information 5 is a text that is a result of recognizing the voice 2 of the speaker 1a, and is a string of speech words obtained by estimating contents of speech.

In Step 102, the face recognition section 61 performs face recognition on the speaker 1a. For example, the face of the speaker 1a is detected in an image (an image in the field of view of the receiver 1b) captured using the face recognition camera 28b of the smart glasses 20b. The position and the region of the face of the speaker 1a on the display screen 6b are estimated on the basis of a result of the detection.

Note that, depending on, for example, an orientation of the face of the receiver 1b (a pose of the smart glasses 20b), it may be difficult to perform face recognition on the speaker 1a using the image captured using the face recognition camera 28b.

In Step 103, the expression analyzer 63 performs expression analysis using the image of the speaker 1a. For example, a degree of smiling and a type of feeling are estimated with respect to the face of the speaker 1a that is detected in Step 102.

In Step 104, the feeling analyzer 64 performs feeling analysis using the voice 2 of the speaker 1a. For example, the voice 2 of the speaker 1a is collected, and the type of feeling of the speaker 1a when the speaker 1a produces the voice 2 is estimated from sound data.

Moreover, for example, a process of detecting a gesture of the speaker 1a that is performed by the gesture recognition section 65 may be performed in parallel as the various recognition processes performed on the speaker 1a.

The information estimated in, for example, Steps 103 and 104 is output to the output controller 67 as feeling information that indicates the feeling of the speaker 1a. Note that there is a possibility that the feeling and the like of the speaker 1a will not be estimated.

Next, the text information 5 (a string of speech words) corresponding to a recognition result of sound recognition is displayed (Step 105). The text information 5 output by the sound recognition section 62 is output to the smart glasses 20b through the output controller 67 and displayed on the display 30b viewed by the receiver 1b. Likewise, the text information 5 is output to the smart glasses 20a through the output controller 67 and displayed on the display 30a viewed by the speaker 1a.

Note that the text information 5 displayed here may also be a string of words that is an intermediate result of sound recognition, or a wrong string of words that is erroneously recognized by sound recognition.

Next, a process of determining the viewing state of the receiver 1b with respect to the text information 5 displayed on the smart glasses 20b is performed (Step 106). Specifically, on the basis of line-of-sight information regarding the line of sight of the receiver 1b, it is determined whether the receiver 1b is in a state of having viewed the text information 5. This makes it possible to detect, for example, a state in which the receiver 1b has not viewed the text information 5 by being affected by, for example, the expression of the speaker 1a.

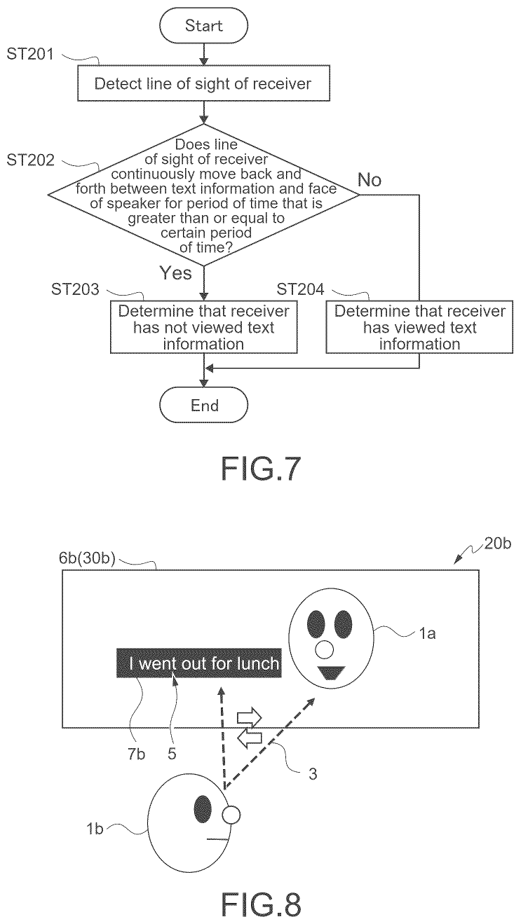

The determination process related to the viewing state of the receiver 1b will be described in detail later with reference to, for example, FIGS. 7 to 13.

When it has been determined that the receiver 1b is not in a state of having viewed the text information 5, that is, when it has been determined that the receiver 1b is in a state of not having viewed the text information 5 (No in Step 106), the output controller 67 performs setting with respect to check-needed information (Step 107).

Here, when it has been determined that the receiver 1b is in a state of not having viewed the text information 5, the text information 5 displayed on the smart glasses 20b is set to be check-needed information. In other words, the check-needed information is unread text information 5 considered not to have been read by the receiver 1b.

For example, the text information 5 is treated by being divided into speech-basis strings of words (phrases) based on, for example, an interval of no voice 2 or a speech partition. Alternatively, the text information 5 may be divided into phrases on the basis of a sentence or word depending on the meaning of contents of speech. In Step 107, a phrase displayed on the smart glasses 20b at a timing at which it is determined that the receiver 1b has not viewed the text information 5 is set to be the check-needed information.

A method for setting the text information 5 as check-needed information is not limited. Typically, a string of words of a phrase corresponding to check-needed information is copied, and temporarily stored (buffered) as text data. Alternatively, sound data corresponding to a phrase that has not been viewed by the receiver 1b is copied/buffered, and check-needed information may be newly generated from the sound data. Further, check-needed information may be generated using sound data collected when it is determined that the receiver 1b is in a state of not having viewed the text information 5.

The speaker 1a is informed of check-needed information (the text information 5 not having been viewed by the receiver 1b) (Step 108). In this case, for example, the text information 5 set to be check-needed information is highlighted to be displayed on the smart glasses 20a used by the speaker 1a. This makes it possible to inform the speaker 1a of the text information 5 (check-needed information) not having been read by the receiver 1b. This enables the speaker 1a to speak, for example, contents of check-needed information again.

Note that a process of informing the speaker 1a that the receiver 1b is in a state of not having viewed the text information 5 may be performed instead of the process of displaying check-needed information to the speaker 1a. In this case, the speaker 1a speaks, for example, contents of most recent speech again, and this enables the speaker 1a to inform again the receiver 1b of contents of speech that have not been viewed by the receiver 1b.

Note that the process of Step 108 does not necessarily have to be performed.

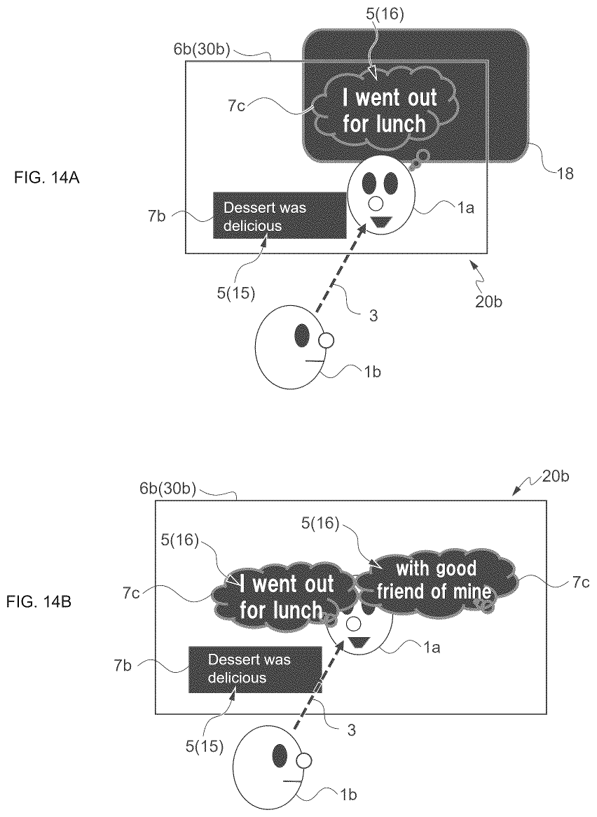

The check-needed information (the text information 5 not having been viewed by the receiver 1b) is displayed to the receiver 1b (Step 109). Here, the output controller 67 displays, on the display screen 6b viewed by the receiver 1b, a string of words that indicates contents of check-needed information and a string of words that indicates the text information 5 obtained by converting the latest speech into a text, such that these strings of words do not overlap (refer to, for example, FIGS. 13 and 14).

From among the displayed strings of words, check-needed information remains displayed until the check-needed information is determined to be unnecessary information. Thus, a plurality of pieces of check-needed information may be displayed on the display screen 6b.

Further, the text information 5 obtained by converting the latest speech into a text is updated as appropriate following speech of the speaker 1a. The text information 5 obtained by converting the latest speech into a text may be hereinafter referred to as updated text information.

In the present embodiment, when the receiver 1b is in a state of not having viewed the text information 5, the text information 5 not having been viewed by the receiver 1b is set to be check-needed information, and the check-needed information remains displayed on the smart glasses 20b used by the receiver 1b, as described above.

For example, when the speaker 1a continuously speaks, the pieces of text information 5 (pieces of updated text information) indicating contents of the speech are successively displayed on the smart glasses 20b. This update of the text information 5 continues regardless of whether the receiver 1b has viewed the text information 5. Thus, the text information 5 (updated text information) not having been viewed by the receiver 1b may be deleted before being checked by the receiver 1b in order to display next contents of speech.

Thus, in the present embodiment, the text information 5 not having been viewed by the receiver 1b is displayed as check-needed information independently of updated text information. This enables the receiver 1b to easily check, on the display screen 6b, the text information 5 that the receiver 1b missed while checking, for example, the expressions of the speaker 1a.

Next, it is determined whether check-needed information displayed on the smart glasses 20b includes unnecessary information (Step 110). Here, the unnecessary information determining section 68 determines a determination condition that is to be satisfied by unnecessary information with respect to the displayed check-needed information.

The determination conditions are conditions regarding, for example, whether the receiver 1b has checked check-needed information and a period of time for which check-needed information is displayed (refer to, for example, FIG. 15).

When there is check-needed information that satisfies the determination condition, it is determined that there is unnecessary information (Yes in Step 110). In this case, display of the check-needed information determined by the output controller 67 to be unnecessary information is deleted (Step 111). Note that check-needed information determined to not be unnecessary information remains displayed.

As described above, the output controller 67 deletes check-needed information determined to be unnecessary information. This makes it possible to avoid blocking the field of view of the receiver 1b due to an increase in the number of pieces of check-needed information displayed.

On the other hand, when there is not any check-needed information that satisfies the determination condition, it is determined that there is no unnecessary information (No in Step 110). In this case, it is determined that there is not any check-needed information to be deleted. The process returns to the parallel process of Steps 101 to S104, and a next loop process is started.

Returning to Step 106, when it has been determined that the receiver 1b is in a state of having viewed the text information 5 (Yes in Step 106), it is determined whether feeling information regarding the feeling of the speaker 1a has been detected (Step 112).

For example, when a type and a degree of the feeling of the speaker 1a are estimated using, for example, the expression analysis performed in Step 103, the feeling analysis performed in Step 104, or gesture recognition, it is determined that the feeling information has been detected (Yes in Step 112).

In this case, a process of decorating current text information 5 (updated text information) is performed according to the feeling information regarding the feeling of the speaker 1a (Step 113). Here, the output controller 67 controls a display form such as a font, a color, and a size of the text information 5 such that the feeling of the speaker 1a is represented.

Further, a visual effect is added to a portion situated around current text information (updated text information) according to the feeling information regarding the feeling of the speaker 1a (Step 114). Here, the output controller 67 generates a visual effect that represents the feeling of the speaker 1a, and the generated visual effect is displayed on a portion situated around the text information 5.

The process of decorating the text information 5 and the process of adding a visual effect will be described in detail later with reference to, for example, FIG. 16.

On the other hand, when the type and the degree of the feeling of the speaker 1a are not estimated in Steps 103 and 104, it is determined that the feeling information has not been detected (No in Step 112). In this case, in this case, the decoration of the text information 5 and the addition of a visual effect are not performed. The process returns to the parallel process of Steps 101 to S104, and a next loop process is started.

### [Presentation of Text Information to Receiver]

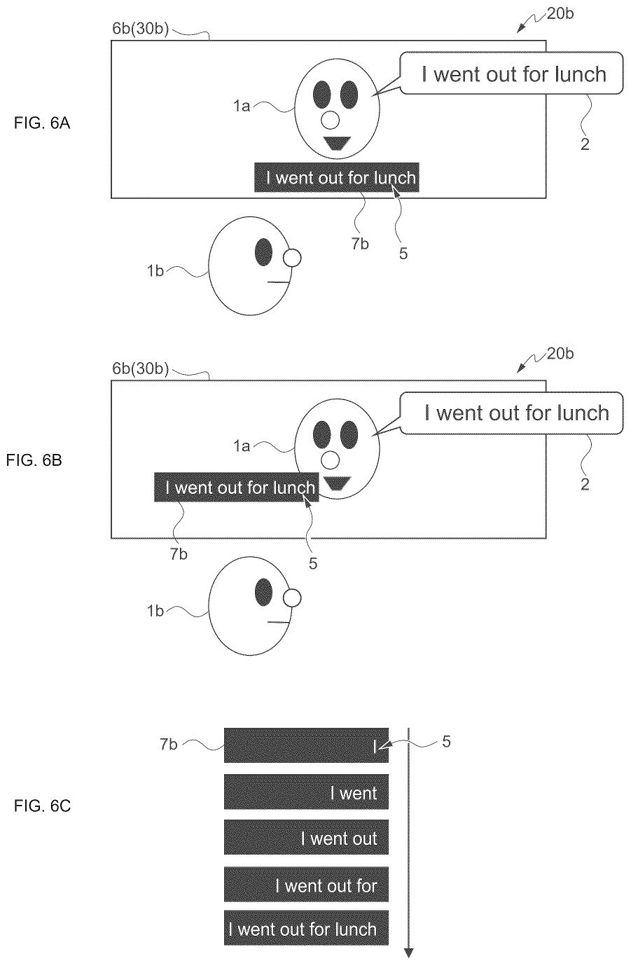

FIG. 6 schematically illustrates examples of presenting text information to the receiver 1b. A and B of FIG. 6 schematically illustrate examples of the display screen 6b displayed on the display 30b by the process of displaying the text information 5 to the receiver 1b that is performed in Step 105 illustrated in FIG. 5.

Here, the speaker 1a speaks “I went out for lunch”, and the voice 2 corresponding to the speech is converted into a text using sound recognition. Then, a string of words that indicates “I went out for lunch” is displayed in the rectangle object 7b (the text display region 10b) on the display screen 6b in the form of a text (the text information 5) that corresponds to a result of sound recognition performed on the speaker 1a.

In the example illustrated in A of FIG. 6, the text information 5 (the object 7b) is displayed at a fixed position set on the display screen 6b. Here, the text information 5 is displayed in the middle of a lower portion on the display screen 6b. Moreover, the placement of the text information 5 (the object 7b) is not limited, and, for example, the object 7b may be placed at any position such as in a center portion, in the middle of an upper portion, in the middle on the left, in the middle on the right, on the upper left, on the lower left, on the upper right, or on the lower right on the display screen 6b. Further, for example, a display position for the object 7b may be adjustable by the receiver 1b as appropriate.

This results in displaying the text information 5 at a fixed position on the display screen 6b. This enables the receiver 1b to check the text information 5 at a fixed position regardless of, for example, a position of the speaker 1a or a pose of the receiver 1b, and to stably view the text information 5.

In the example illustrated in B of FIG. 6, the text information 5 (the object 7b) is displayed at a position based on the position of the face of the speaker 1a on the display screen 6b. In this process, the display position for the text information 5 is calculated as appropriate on the basis of, for example, a detection result obtained by detecting the face of the speaker 1a in Step 102 illustrated in FIG. 5. Here, the text information 5 is displayed in a portion that corresponds to a left portion of a mouth of the speaker la. Moreover, the object 7b may be placed in a portion that corresponds to, for example, a right portion or a lower portion of the mouth of the speaker 1a. This results in clearly indicating that, for example, the text information 5 is information provided from the mouth of the speaker 1a and thus in being able to present the text information 5 in an easy-to-understand way. Further, the receiver 1b can check the expression of the speaker 1a and text information without moving the line of sight 3 broadly.

For example, when the text information 5 is displayed on the display screen 6b in a fixed manner, the face of the speaker 1a may be distant from the text information 5, or the face of the speaker 1a may overlaps the text information 5 depending on the viewing position or the movement. This may result in difficulty in checking the face of the speaker 1a and the text information 5 at the same time. In such a case, the text information 5 is displayed in a portion situated around the face of the speaker 1a, as illustrated in B of FIG. 6. This makes it possible to easily check both the face of the speaker 1a and the text information 5.

C of FIG. 6 schematically illustrates an example of displaying the text information 5 in the object 7b. As illustrated in C of FIG. 6, a string of words included in the text information 5 is displayed from a right end to a left end of the object 7b. When, for example, a string of words that indicates “I went out” is displayed, “I” is displayed at the right end of the object 7b first. Next, “I” moves to the left by a word, and “went” is displayed at the right end of the object 7b. Next, “I” and “went” each move to the left by a word, and “out” is displayed at the right end of the object 7b.

As described above, a string of words is presented from the right to the left, and this enables the receiver 1b to understand contents of the string of words only by seeing the right end of the object 7b with hardly moving the line of sight 3. Further, there is no need to follow the latest word with the eyes even when the speech is long. This makes it easy to check a string of words. This makes it possible to sufficiently reduce burdens imposed on the receiver 1b when the receiver 1b reads the text information 5.

Note that how to present a string of words is not limited, and, for example, a method for displaying the string of words from the left to the right may be adopted.

### [Determination Process Related to Viewing State of Receiver]

The determination process related to the viewing state of the receiver 1b is specifically described below, the determination process being performed in Step 106 illustrated in FIG. 5.

FIG. 7 is a flowchart illustrating an example of the determination process related to the viewing state of the receiver 1b. FIG. 8 is a schematic diagram used to describe the determination process illustrated in FIG. 7. In the determination process described with reference to FIGS. 7 and 8, the determination process related to the viewing state is performed on the basis of a duration of a back-and-forth movement of the line of sight 3 of the receiver 1b between the face of the speaker 1a and text information.

When the face of the speaker 1a is viewed on the display screen 6b viewed by the receiver 1b, as illustrated in, for example, FIG. 8, the receiver 1b can check both the expression of the speaker 1a and the text information 5. When the receiver 1b has the expression of the speaker 1a on his/her mind in particular, the line of sight 3 of the receiver 1b may frequently move back and forth between the text information 5 and the face of the speaker 1a, in order to check the face of the speaker 1a while keeping his/her mind on the text information 5.

When the line of sight 3 of the receiver 1b continuously moves back and forth for a period of time that is greater than or equal to a certain period of time, it is determined that the receiver 1b is in a state of not concentrating on the text information 5 and of not having viewed the text information 5. Note that, when the period of time for which the back-and-forth movement is performed does not exceed the certain period of time, the receiver 1b may be in a state of, for example, having checked the expression of the speaker 1a only for a moment, and it is determined that the receiver 1b is in a state of having viewed the text information 5.

First, the line-of-sight detector 60 detects the line of sight 3 of the receiver 1b, as illustrated in FIG. 7 (Step 201). Here, positional coordinates for a viewpoint of the receiver 1b on the display screen 6b are assumed to be detected as the line of sight 3 of the receiver 1b.

Next, it is determined whether the viewpoint of the receiver 1b continuously moves back and forth between the text information 5 and the face of the speaker 1 for a period of time that is greater than or equal to a certain period of time (Step 202). For example, a duration of the back-and-forth movement is measured, with a timing at which the viewpoint of the receiver 1b moves from the text display region 10b in which the text information 5 is displayed to the region of the face of the speaker 1 being a start time. Here, for example, a state in which the viewpoint of the receiver 1b moves back and forth between the text display region 10b and the region of the face of the speaker 1 without remaining in one region for a period of time that is greater than a specified period of time, is detected as a back-and-forth movement.

When the duration of the back-and-forth movement is greater than or equal to the certain period of time (Yes in Step 202), the receiver 1b is not concentrating on the text information 5, and it is determined that the receiver 1b has not viewed the text information 5 (Step 203).

Conversely, when the duration of the back-and-forth movement is less than the certain period of time (No in Step 202), it is determined that the receiver 1b has viewed the text information 5 (Step 203).

This makes it possible to detect a state in which the receiver 1b is not concentrating on the text information 5 since the receiver 1b is checking the face of the speaker 1a.

Note that, in Step 202 illustrated in FIG. 7, the number of times of the back-and-forth movement may be determined instead of the duration of the back-and-forth movement. In other words, the determination process related to the viewing state may be performed on the basis of the number of times of the back-and-forth movement of the line of sight 3 of the receiver 1b between the face of the speaker 1a and text information.

In this case, when the number of times of the back-and-forth movement is greater than or equal to a certain number of times, it is determined that the receiver 1b has not viewed the text information 5. When the number of times of the back-and-forth movement is less than the certain number of times, it is determined that the receiver 1b has viewed the text information 5.

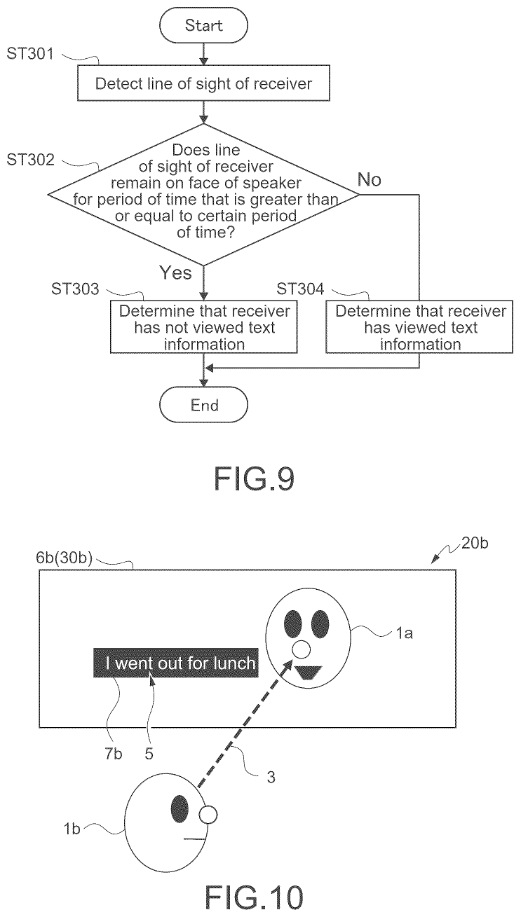

FIG. 9 is a flowchart illustrating another example of the determination process related to the viewing state of the receiver 1b. FIG. 10 is a schematic diagram used to describe the determination process illustrated in FIG. 9. In the determination process described with reference to FIGS. 9 and 10, the determination process related to the viewing state is performed on the basis of a remaining period of time for which the line of sight 3 of the receiver 1b remains on the face of the speaker 1a.

It is assumed that the face of the speaker 1a is viewed on the display screen 6b viewed by the receiver 1b, as illustrated in, for example, FIG. 10. When, for example, the receiver 1b is concentrating on the expression of or the movement of the mouth of the speaker 1a, the line of sight 3 of the receiver 1b is oriented toward the face of the speaker 1a in order to check the face of the speaker 1a. In other words, the line of sight 3 of the receiver 1b remains on the face of the speaker 1a without returning to, for example, the text information 5.

When the line of sight 3 of the receiver 1b remains on the face of the speaker 1a for a period of time that is greater than or equal to a certain period of time, as described above, the receiver 1b is concentrating on the face of the speaker 1a, and it is determined that the receiver 1b is in a state of not having viewed the text information 5. Note that, when the remaining period of time does not exceed the certain period of time, the receiver 1b may be in a state of, for example, having checked the expression of the speaker 1a only for a moment, and it is determined that the receiver 1b is in a state of having viewed the text information 5.

First, the line-of-sight detector 60 detects the line of sight 3 of the receiver 1b, as illustrated in FIG. 9 (Step 301). Here, positional coordinates for the viewpoint of the receiver 1b on the display screen 6b are assumed to be detected, as in the case of Step 201 illustrated in FIG. 7.

Next, it is determined whether the viewpoint of the receiver 1b remains on the face of the speaker 1 for a period of time that is greater than or equal to a certain period of time (Step 302). For example, a period of time that elapses before the viewpoint of the receiver 1b leaves the region of the face of the speaker 1a is measured as the remaining period of time, with a timing at which the viewpoint of the receiver 1b enters the region of the face of the speaker 1a being a start time.

When the remaining period of time for which the viewpoint of the receiver 1b remains on the face of the speaker 1 is greater than or equal to the certain period of time (Yes in Step 202), the receiver 1b is concentrating on the face of the speaker 1a, and it is determined that the receiver 1b has not viewed the text information 5 (Step 303).

Conversely, when the remaining period of time is less than the certain period of time (No in Step 302), it is determined that the receiver 1b has viewed the text information 5 (Step 303).

This makes it possible to detect a state in which the receiver 1b is concentrating on the face of the speaker 1a and not concentrating on the text information 5.

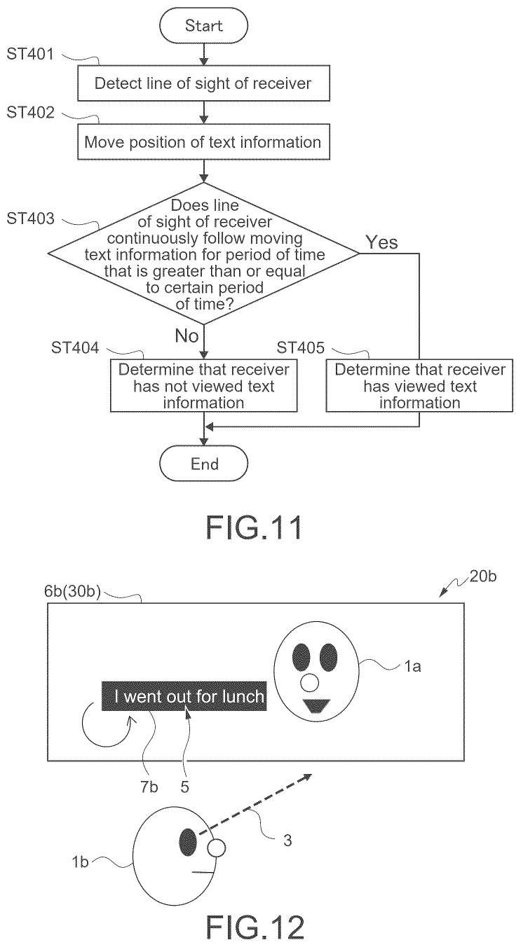

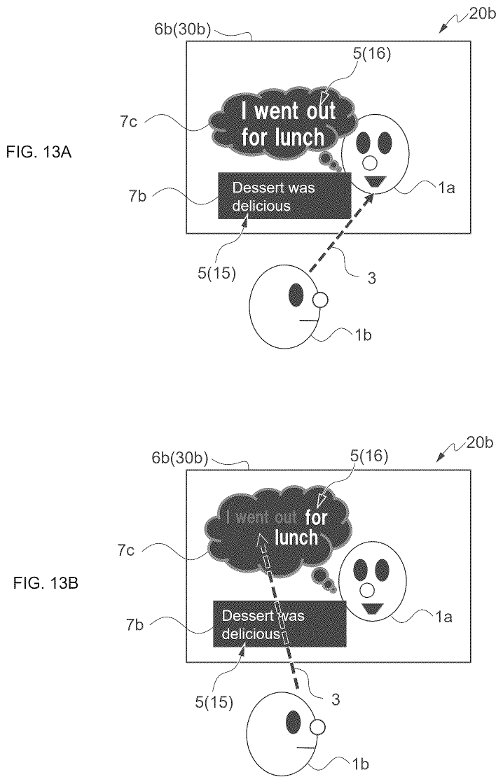

FIG. 11 is a flowchart illustrating another example of the determination process related to the viewing state of the receiver 1b. FIG. 12 is a schematic diagram used to describe the determination process illustrated in FIG. 11. In the determination process described with reference to FIGS. 11 and 12, the text information 5 is displayed on the smart glasses 20b (the display screen 6b) used by the receiver 1b in a state in which the text information 5 is moving. Then, the determination process related to the viewing state is performed on the basis of a following period of time for which the line of sight 3 of the receiver 1b follows the moving text information 5.

It is assumed that the text information 5 (the object 7b) is displayed on the display screen 6b such that the text information 5 is moving along a specified path, as illustrated in, for example, FIG. 12. For example, when the receiver 1b is paying attention to the text information 5, the receiver 1b may follow the moving text information 5 with his/her eyes, that is, the line of sight 3 of the receiver 1b may move to follow the text information 5. Conversely, when the receiver 1b is not paying attention to the text information 5, the line of sight 3 of the receiver 1b may move independently of the movement of the text information 5.

When, for example, the following period of time for which the line of sight 3 of the receiver 1b follows the text information 5 is greater than or equal to a certain period of time, it is determined that the receiver 1b is concentrating on the text information 5. Conversely, when the following period of time does not exceed the certain period of time, there is a possibility that, for example, the receiver 1b is not paying attention to the text information 5, and it is determined that the receiver 1b is in a state of not having viewed the text information 5.

First, the line-of-sight detector 60 detects the line of sight 3 of the receiver 1b, as illustrated in FIG. 9 (Step 401). Here, positional coordinates for the viewpoint of the receiver 1b on the display screen 6b are assumed to be detected, as in the case of Step 201 illustrated in FIG. 7.

Next, the output controller 67 starts performing a process of displaying the text information 5 on the display screen 6b while moving the text information 5 (Step 402). In the present embodiment, the movement of the text information 5 is started at a timing at which the receiver 1b starts looking at the text information 5, and the text information 5 continuously moves for a certain period of time. During this period of time, a period of time for which the line of sight 3 of the receiver 1b follows the text information 5 is determined.

The text information 5 starts moving at the timing at which the receiver 1b starts looking at the text information 5, as described above. This makes it possible to determine, with certainty, whether the receiver 1b is paying attention to the text information 5. Further, a period of time for which the text information 5 moves is reduced. This makes it possible to determine the viewing state of the receiver 1b with hardly causing the receiver 1b to experience, for example, a feeling of exhaustion caused due to the movement of the line of sight 3.

Next, it is determined whether the viewpoint of the receiver 1b continuously follows the text information 5 for a period of time that is greater than or equal to a certain period of time (Step 403). For example, a period of time for which the viewpoint of the receiver 1b remains in the moving text display region 10b is measured as the following period of time, with a timing at which the viewpoint of the receiver 1b enters the text display region 10b in which the text information 5 is displayed being a start time.

When the following period of time for which the viewpoint of the receiver 1b follows the moving text information 5 is less than the certain period of time (No in Step 403), the receiver 1b is not concentrating on the moving text information 5, and it is determined that the receiver 1b has not viewed the text information 5 (Step 404).

Conversely, when the following period of time is greater than or equal to the certain period of time (Yes in Step 403), the receiver 1b follows the moving text information 5 with his/her eyes, and it is determined that the receiver 1b has viewed the text information 5 (Step 404).

This makes it possible to detect whether the receiver 1b is concentrating the text information 5 itself.

Further, for example, the above-described processes described with reference to FIGS. 7 to 10 are processes that can be performed when the face of the speaker 1a has been detected. On the other hand, the process being described with reference to FIGS. 11 and 12 and using whether the line of sight 3 of the receiver 1b follows the text information 5, is a process that makes it possible to determine the viewing state of the receiver 1b regardless of whether the face of the speaker 1a is detected. This makes it possible to determine the viewing state of the receiver 1b with certainty even when, for example, the face of the speaker 1a is not detected.

A method for setting determination thresholds used to determine, for example, the duration of or the number of times of the back-and-forth movement of the line of sight of the receiver 1b, the remaining period of time for which the line of sight of the receiver 1b remains on the face of the speaker 1a, and the following period of time for which the line of sight of the receiver 1b follows text information, is not limited. For example, respective thresholds may be set as appropriate such that a state in which the receiver 1b has not viewed the text information 5 can be determined properly.

Further, the determination thresholds used to perform the determination processes related to the viewing state may be adjusted according to, for example, a state of the sound recognition process. In the present embodiment, the viewing state determining section 66 changes, according to a speed at which the text information 5 is updated, the determination threshold used to perform the determination process related to the viewing state.

When, for example, it takes a long time to perform the sound recognition process, a period of time from the speaker 1a starting speaking to a string of words (the text information 5) that is a recognition result being displayed, is long. In other words, the speed at which the text information 5 is updated may be slow, and a string of words may be slow to be updated. When the text information 5 is not updated, as described above, the receiver 1b may view the face of the speaker 1a more frequently.

When, for example, adjustment of a determination threshold is not performed in such a state, the probability of it being determined that the text information 5 has not been viewed may be higher despite the fact that the receiver 1b has viewed the text information 5 completely.

Thus, in the present embodiment, the determination threshold is dynamically changed when a result of sound recognition is not updated (when an update speed is decreased), such that it is less likely to be determined that the receiver 1b is in a state of not having viewed the text information 5. In other words, the determination threshold is adjusted such that the probability of it being determined that the receiver 1b has not viewed the text information 5 is made lower.

When, for example, the update speed is low, the determination threshold related to the duration of or the number of times of the back-and-forth movement is set to exhibit a value larger than a usual value. Further, the determination threshold related to the remaining period of time for which the line of sight of the receiver 1b remains on the face of the speaker 1a, and the determination threshold related to the following period of time for which the line of sight of the receiver 1b follows text information are also each set to exhibit a larger value when the update speed is low. This results in it being less likely to be determined that the receiver 1b has not viewed the text information 5. This makes it possible to avoid displaying, as check-needed information, information that has been completely checked by the receiver 1b.

### [Presentation of Check-Needed Information to Receiver]

A process of presenting check-needed information to the receiver 1b that is performed in Step 109 illustrated in FIG. 5 is specifically described below. FIGS. 13 and 14 schematically illustrate examples of the process of presenting check-needed information to the receiver 1b. In each of A and B of FIG. 13 and A and B of FIG. 14, two kinds of the text information 5 that are updated text information 15 and check-needed information 16 are displayed on the display screen 6b viewed by the receiver 1b.

The updated text information 15 is displayed within the rectangle object 7b. Further, the check-needed information 16 is displayed within a cloud-shaped object 7c.

Here, it is assumed that the speaker 1a speaks “I went out for lunch”, and thereafter the speaker 1a speaks “Dessert was delicious”.

When the text information 5 indicating contents of speech that are “I went out for lunch” from among the speech of the speaker 1a is displayed as the updated text information 15 (is displayed within the object 7b), it is determined that the receiver 1b is in a state of not having viewed the text information 5. In this case, the text information 5 including a string of words that indicates “I went out for lunch” is set to be the check-needed information 16.

In scenes illustrated in FIGS. 13 and 14, the text information 5 indicating “I went out for lunch”, which is the most recent speech, is displayed as the check-needed information 16 while the latest text information 5 (the updated text information 15) indicating “Dessert was delicious” is being displayed.

A of FIG. 13 schematically illustrates an example of a process of displaying the check-needed information 16 above the updated text information 15. Here, the object 7c in which the check-needed information 16 is displayed is placed above the object 7b in which the updated text information 15 is displayed.

When, for example, a plurality of pieces of check-needed information 16 is set, the pieces of check-needed information 16 are stacked to be displayed above the updated text information 15 in order of newer setting from the updated text information 15. Thus, the latest check-needed information 16 is displayed immediately above the updated text information 15 at all times.

In the example illustrated in A of FIG. 13, the check-needed information 16 is stacked to be displayed above the newly displayed text information 5 (the updated text information 15) on the smart glasses 20b (the display screen 6b) used by the receiver 1b, as described above.

As described above, the check-needed information 16 is stacked for each speech to be additionally presented above the updated text information 15 corresponding to a string of speech words that indicates the latest contents of speech. This enables the receiver 1b to easily check unchecked text information 5 in order of newer setting. For example, it is often the case that the text information 5 not having been viewed in the most recent speech is more important than the old text information 5 in order to understand current contents of speech. In the display method illustrated in A of FIG. 13, the most recent check-needed information 16 is displayed most closely to the updated text information 15. This enables the receiver 1b to easily check, for example, missed contents of speech quickly.

Further, the check-needed information 16 is displayed using a font, a color, a size, and a background style (the object 7c) that are different from those used for the updated text information 15, as illustrated in A of FIG. 13. In other words, the output controller 67 displays the check-needed information 16 in a display mode that is different from a display mode used for other text information 5 (the updated text information 15) that is displayed on the same display screen 6b as the check-needed information 16.

In A of FIG. 13, the font of the check-needed information 16 is changed to a font that is thicker than a font of the updated text information 15. Further, the color of the check-needed information 16 is changed to a color that is more noticeable than a color of the updated text information 15. When, for example, the updated text information 15 is in white, the check-needed information 16 is displayed in, for example, yellow. Further, the check-needed information 16 is set larger in size than the updated text information 15. Further, with respect to the background style of the check-needed information 16, the cloud-shaped object 7c is used for the check-needed information 16 in consideration of the rectangle object 7b in which the updated text information 15 is displayed.

As described above, the display mode for the check-needed information 16 is made different from the display mode for the updated text information 15. This enables the receiver 1b to distinguish the check-needed information 16 from the updated text information 15 with certainty. Further, the appropriate setting of the display mode for the check-needed information 16 makes it possible to highlight and display a string of words that is to be checked by the receiver 1b.

B of FIG. 13 schematically illustrates an example of control performed on display of a string of words of the check-needed information 16. Here, a string of words, from among strings of words included in the check-needed information 16, that has been read by the receiver 1b is determined. Then, the string of words having been read by the receiver 1b and a string of words that has not been read by the receiver 1b are displayed in different display modes. In other words, display is performed such that what has been read by the receiver 1b is distinguished from what has not been read by the receiver 1b.

In the process of determining a string of words that has been read by the receiver 1b, it is determined, for example, whether the line of sight 3 of the receiver 1b is oriented toward (the viewpoint of the receiver 1b enters) a text region set for a string of words.

Here, for example, the text region refers to a region formed by surrounding a word or a character (such as the alphabet, figures, kanji, hiragana, or katakana). Alternatively, a word- or syllable-based region including a word or a syllable may be set to be the text region. Alternatively, the above-described text display region 10b may be used as the text region. When the line of sight 3 of the receiver 1b is oriented toward (the viewpoint of the receiver 1b enters) such a text region, it is determined that a character or a word in the text region has been read by the receiver 1b.

In B of FIG. 13, it is detected that the line of sight 3 of the receiver 1b is oriented toward a text region set for each word in “I went out” (or a text region set for the words “I went out”) from among strings of words of the check-needed information 16 indicating “I went out for lunch”. In this case, “I went out” is determined to be a read string of words that has been read by the receiver 1b. Note that other strings of words are determined to be unread strings of words that have not been read by the receiver 1b.

For example, a string of words may be set to be a read string of words when the viewpoint of the receiver 1b enters a text region for the string of words at least once. Alternatively, when the viewpoint of the receiver 1b remains in a region for a period of time that is greater than or equal to a certain period of time, a string of words for which the region is set may be set to be a read string of words. Moreover, a method for determining a read string of words (an unread string of words) is not limited.

With respect to a string of words (here, “I went out”) that has been read by the receiver 1b, the color of the words is changed to a color close to a color of the background style, and adjustment is performed to make the size of the words smaller. This results in a read string of words becoming unnoticeable, and in being able to relatively highlight and display an unread string of words. This enables the receiver b to easily recognize a string of words to be read, and to check the check-needed information 16 efficiently.

In A of FIG. 14, a display position for the check-needed information 16 is set according to a state around the receiver 1b. Specifically, the check-needed information 16 is displayed on a background viewed through the smart glasses 20b used by the receiver 1b such that the check-needed information 16 overlaps a portion of the background that makes the display of the check-needed information 16 noticeable.

Here, the portion making the display of the check-needed information 16 noticeable (hereinafter referred to as an overlap-target region 18) is, for example, a region in which the check-needed information 16 can be displayed with high contrast. A of FIG. 14 schematically illustrates a black region that corresponds to the overlap-target region 18 viewed through the display screen 6b.

For example, a dark-color region that is situated around the receiver 1b and viewed through the smart glasses 20b is detected as the overlap-target region 18 when, for example, the communication system 100 is used outdoors. For example, the overlap-target region 18 is detected by, for example, an image recognition process being performed on an image captured using the face recognition camera 28b. Then, the text information 5 is displayed to overlap the dark-color overlap-target region 18. Here, for example, the color of the check-needed information 16 may be set to exhibit a high contract to the color of the overlap-target region 18. This makes it possible to make the check-needed information 16 noticeable even in a relatively bright place such as outdoors. This makes it possible to present the check-needed information 16 to the receiver 1b in a state of emphasizing the presence of the check-needed information 16, and to encourage the receiver 1b to check the check-needed information 16.

In B of FIG. 14, the display position for the check-needed information 16 is set according to the position of the face of the speaker 1a. Specifically, the check-needed information 16 is displayed to overlap the face of the speaker 1a.

Here, it is assumed that the speaker 1a speaks “I went out for lunch” “with a good friend of mine” and thereafter the speaker 1a speaks “Dessert was delicious”. From among the speech of the speaker 1a, the string of words “I went out for lunch” and the string of words “with a good friend of mine” are set to be the pieces of check-needed information 16.

In B of FIG. 14, the two pieces of check-needed information 16 are displayed to overlap the face of the speaker 1a purposely. For example, the string of words “I went out for lunch” is displayed, and a portion of the face of the speaker 1a becomes invisible. Thereafter, the string of words “with a good friend of mine” is further displayed, and the face of the speaker 1a becomes less visible. As described above, a display process that includes purposely presenting, in a portion situated around the face of the speaker 1a, the check-needed information 16 not having been viewed by the receiver 1b, is performed.

Consequently, the check-needed information 16 becomes an obstacle that makes the face of the speaker 1a invisible. Thus, for example, this display process serves as a mechanism that causes the receiver 1b wanting to check the expression of the speaker 1a to feel like removing the check-needed information 16 corresponding to an obstacle. This makes it possible to cause the receiver 1b to check the check-needed information 16 spontaneously.

Further, the check-needed information 16 may overlap according to properties of an object situated around the receiver 1b. When, for example, a container such as a basket or a dish is situated around the receiver 1b, a process of displaying the check-needed information 16 such that the check-needed information 16 overlaps the container in a state of being in the container, is performed. This makes it possible to cause the virtually displayed check-needed information 16 to appear to be accumulated in a container in a real space, and thus to intuitively inform the receiver 1b that information to be checked is accumulated. Moreover, a process of displaying the check-needed information 16 in a state of being attached to a refrigerator or a wall surface, or a process of displaying the check-needed information 16 in a state of fitting a page of a document or a book may be performed.

A method for presenting the check-needed information 16 to the receiver 1b is not limited, and, for example, any display method that enables, for example, display that causes the receiver 1b to feel like checking the check-needed information 16 or display that makes the check-needed information 16 noticeable may be used.

### [Deletion of Display of Check-Needed Information]

FIG. 15 schematically illustrates an example of a process of deleting display of the check-needed information 16. FIG. 15 schematically illustrates deletion of one of two pieces of check-needed information 16 displayed on the display screen 6b viewed by the receiver 1b.

A series of processes performed in Steps 110 and 111 illustrated in FIG. 5 to delete the check-needed information 16 is specifically described below.

Here, it is assumed that the speaker 1a speaks “I went out for lunch”, “Dessert was delicious”, and “Let's go there again next Sunday” in this order.

From among the speech of the speaker 1a, the text information 5 indicating “I went out for lunch” is set to be check-needed information 16a, and the text information 5 indicating “Dessert was delicious” is set to be check-needed information 16b. Further, the latest text information 5 indicating “Let's go there again next Sunday” is displayed as the updated text information 15. In this case, the check-needed information 16b and the check-needed information 16a are stacked to be displayed above the updated text information 15 on the display screen 6b in this order from the updated text information 15.

When the check-needed information 16 is displayed, first, the unnecessary information determining section 68 determines unnecessary information from among the check-needed information 16 displayed on the display screen 6b (refer to Step 110 illustrated in FIG. 5). The determination process is performed to determine, for example, whether the receiver 1b has checked the displayed check-needed information 16 and a period of time for which the displayed check-needed information 16 is displayed. Further, the output controller 67 deletes, from the display screen 6b, display of the check-needed information 16 determined to be unnecessary (refer to Step 111 illustrated in FIG. 5).

In the present embodiment, the unnecessary information determining section 68 determines, on the basis of line-of-sight information regarding the line of sight of the receiver 1b, whether the receiver 1b has checked the check-needed information 16. Then, the check-needed information 16 determined to have been checked by the receiver 1b is determined to be unnecessary information. When it is determined whether the receiver 1b has checked the check-needed information 16, a region of the check-needed information 16 (such as a text region of a string of words of the check-needed information 16) and a position of the line of sight (the viewpoint) of the receiver 1b are referred to.

When, for example, the viewpoint of the receiver 1b enters the region of the check-needed information 16, or when the viewpoint of the receiver 1b remains in the region of the check-needed information 16 for a period of time that is greater than or equal to a certain period of time, this check-needed information 16 is determined to have been checked by the receiver 1b and thus determined to be unnecessary information.

Further, when, for example, the viewpoint of the receiver 1b moves back and forth between the region of the check-needed information 16 and the region of the face of the speaker 1b for a period of time that is greater than or equal to a certain period of time, or the number of times that is greater than or equal to a certain number of times, this check-needed information 16 is determined to have been checked by the receiver 1b and thus determined to be unnecessary information.

The line of sight 3 of the receiver 1b is referred to, as described above, and this makes it possible to determine, as unnecessary information, the check-needed information 16 having been actually viewed by the receiver 1b.

Further, the check-needed information 16 displayed on the smart glasses 20b used by the receiver 1b for a period of time that exceeds a threshold may be determined to be unnecessary information. In other words, the check-needed information 16 for which a certain period of time has elapsed since the check-needed information 16 started being displayed, is determined to be unnecessary information. This results in display of the check-needed information 16 disappearing after the elapse of the certain period of time. This makes it possible to prevent an unnecessarily large number of pieces of check-needed information 16 from being displayed and to prevent the receiver 1b from having difficulty in seeing in the field of view.

Further, a piece of check-needed information 16 displayed on the smart glasses 20b used by the receiver 1b for a longest period of time at a timing at which the number of pieces of check-needed information 16 displayed on the smart glasses 20b used by the receiver 1b exceeds a threshold, may be determined to be a piece of unnecessary information. In other words, when the number of pieces of check-needed information 16 displayed exceeds a certain number, the oldest piece of check-needed information 16 in the displayed pieces of check-needed information 16 is determined to be a piece of unnecessary information. This makes it possible to, for example, successively delete, in a chronological order, pieces of check-needed information 16 situated beyond a space situated above the updated text information 15.

On the left in FIG. 15, the check-needed information 16b toward which the line of sight 3 of the receiver 1b is oriented (that the viewpoint of the receiver 1b enters) is determined to be unnecessary information, and displayed contents (a string of words and the object 7c) of the check-needed information 16b are deleted. For example, there is an increase in a degree of transparency with which the check-needed information 16b is displayed, and the check-needed information 16b is faded out to be deleted. Alternatively, the check-needed information 16b may be deleted by moving outward from the display screen 6b.

When the check-needed information 16 is deleted, the check-needed information 16a moves downward to be displayed immediately above the updated text information 15, as illustrated on the left in FIG. 15. This makes it possible to place unread check-needed information 16 near the updated text information 15, and this enables the receiver 1b to easily check the unread check-needed information 16.

A method for setting the determination thresholds used to determine unnecessary information is not limited. For example, each threshold may be set as appropriate such that, for example, checking of the text information 5 that is performed by the receiver 1b can be properly determined.

Further, the determination threshold used to determine unnecessary information may be adjusted according to the nature of the receiver 1b. In the present embodiment, the unnecessary information determining section 68 changes the determination threshold used to perform a process of the determination related to unnecessary information, according to how frequently the receiver 1b looks at the face of the speaker 1a.

When, for example, the receiver 1b frequently looks at the face of the speaker 1a, it is determined that the receiver 1b tends to lay emphasis on, for example, the expression of the speaker 1a, and thus the determination threshold used to determine the check-needed information 16 as unnecessary information is set low such that the check-needed information 16 can be more easily deleted. This results in not unnecessarily blocking the field of view of the receiver 1b. This makes it possible to provide an environment in which the receiver 1b can sufficiently check both the expression of the speaker 1a and the text information 5.

Further, when, for example, the receiver 1b less frequently looks at the face of the speaker 1a, it is determined that the receiver 1b tends to lay emphasis on the text information 5, and thus the determination threshold used to determine the check-needed information 16 as unnecessary information is set high such that the check-needed information 16 is sufficiently checked. This makes it possible to provide an environment in which the receiver 1b can check, with certainty, the text information 5 that the receiver 1b has missed.

### [Arrangement on Text Information in State in Which Receiver Have Been Viewed Text Information]

The process performed when it is determined that the receiver 1b is in a state of not having viewed the text information 5 (No in Step 106 illustrated in FIG. 5) has been described above. A process performed when the receiver 1b is in a state of having viewed the text information 5 (Yes in Step 106 illustrated in FIG. 5) is described below.

FIG. 16 schematically illustrates examples of a process of decorating text information in a state of having been viewed by the receiver 1b. In each of A and B of FIG. 16, it is determined that the receiver 1b is in a state of having viewed the text information 5, and the updated text information 15 corresponding to the latest result of sound recognition (the text information 5) is displayed on the display screen 6b viewed by the receiver 1b. Here, it is assumed that feeling information that indicates the feeling of the speaker 1a is detected using, for example, expression analysis performed on an image of the speaker 1a and feeling analysis performed on speech of the speaker 1a (Yes in Step 112 illustrated in FIG. 5).

In the present embodiment, when the receiver 1b is in a state of having viewed the text information 5, the output controller 67 performs an arrangement process of arranging the text information 5 (the updated text information 15) according to the feeling information regarding the feeling of the speaker 1a. When, for example, the receiver 1b is in a state of being able to read a string of words of contents of speech, arrangement that represents the feeling of the speaker 1a during speaking is added to display of the string of words. This enables the receiver 1b to estimate the feeling of the speaker 1a for each speech of the speaker 1a even when the receiver 1b is in a state of not having checked, for example, the expression of the speaker 1a.

In A of FIG. 16, the text information 5 is decorated according to feeling information regarding the feeling of the speaker 1a. This is a process performed in, for example, Step 113 illustrated in FIG. 5.

The decoration of the text information 5 is a process of arranging the text information 5 by changing a display form such as a font, a color, and a size of the text information 5. Further, the text information 5 may be arranged by adding a necessary word or reference numeral to a string of words of the text information 5.

In the example illustrated in A of FIG. 16, the speaker 1a speaks “I see”, “Really”, and “Thanks so much”, and each content of speech is converted into a text using sound recognition.

Here, it is assumed to be determined, by expression analysis performed at a timing at which the speaker 1a speaks “Really”, that the expression of the speaker 1a is “smiling”. In this case, a process that includes making the font of the string of words “Really” larger in size, changing the color of the string of words, and adding an exclamation point “!”, is performed. For example, when an initial color of the string of words is white, the word color is changed to a color such as yellow or pink that represents delight.

Further, a certain soundless period of time between strings of words during performing sound recognition may be determined to be an “interval” caused during speech, and “ . . . ” may be displayed with respect to the certain soundless period of time. This makes it possible to represent an interval caused during speech of the speaker 1a, and thus to inform the receiver 1b of, for example, a shade of meaning in speech.

In B of FIG. 16, a visual effect is added to a portion situated around the text information 5 according to feeling information regarding the feeling of the speaker 1a. This is a process performed in, for example, Step 114 illustrated in FIG. 5.

The process of adding a visual effect is, for example, a process of arranging the text information 5 by visualizing the feeling of the speaker 1a using a moving image. Specifically, a visualization object 19 that represents the feeling of the speaker 1a is displayed on the portion situated around the text information 5.

In the example illustrated in B of FIG. 16, the speaker 1a speaks “Wow!”, and contents of the speech are converted into a text using sound recognition. Here, for example, it is assumed to be determined, by feeling analysis performed using sound, that the feeling of the speaker 1a is “delight”. In this case, the visualization object 19 representing “delight” is selected and generated, and the generated visualization object 19 is displayed as a background of the text information 5 indicating “Wow!”.

Moreover, contents of a visual effect are not limited, and, for example, arrangement to move the string of words “Wow!” in the form of animation, or arrangement to cause, for example, flower petals to flutter down may be performed as appropriate. This makes it possible to intuitively inform the receiver 1b of, for example, a shade of meaning in speech even when, for example, the receiver 1b is in a state of not having checked, for example, the expression of the speaker 1a.

In the controller 53 according to the present embodiment, speech of the speaker 1a is converted into a text using sound recognition, and the text is displayed on the smart glasses 20b used by the receiver 1b, as described above. Further, the viewing state of the receiver 1b with respect to the text information 5 is estimated on the basis of line-of-sight information regarding the line of sight of the receiver 1b. Display of the text information 5 is controlled according to the viewing state. This makes it possible to display necessary text information when, for example, the receiver 1b has not viewed the text information 5 completely. This makes it possible to perform communication that enables the receiver to easily check both what the speaker is like and contents of speech of the speaker.

When an application that displays a result of sound recognition to assist communication is used, it is difficult to view a displayed string of words that corresponds to the recognition result as well as, for example, an expression and a gesture of a speaker at the same time. For example, there is a possibility that a feeling of a speaker that is seen from an expression of the speaker and an implicit intension of the speaker will not be grasped if only a string of words corresponding to a recognition result is continuously viewed. Conversely, there is a possibility that conversation will go on before contents of the conversation are caught up when the expression of the speaker is continuously viewed, since a string of words corresponding to a recognition result is not read.

Further, words obtained by sound recognition are often displayed late. Thus, when an expression and a gesture of a speaker are changed while a receiver is reading a late displayed string of words, the receiver will check what the speaker is like, and will not view the string of words.

Further, it may become difficult for the receiver to determine where to restart reading a string of words from once the receiver looks away from the string of words to see the expression of the speaker.

It is difficult to continuously display all of strings of words (strings of speech words) that correspond to recognition results obtained using sound recognition since displays or the like have limited display ranges. Thus, when a string of speech words is actually displayed, there is also a need to perform a process of deleting display of a string of words as appropriate. Thus, when a string of words that a receiver has missed is deleted, the receiver will have no chance to check the missed word.

In the present embodiment, display of the text information 5 is controlled on the basis of the viewing state of the receiver 1b with respect to the text information 5. Consequently, the text information 5 considered to not have been read by the receiver 1b can be displayed as the check-needed information 16 when, for example, the receiver 1b is in a state of not having viewed the text information 5. Further, when the receiver 1b is in a state of having viewed the text information 5, the text information 5 can be arranged according to, for example, the expression of the speaker 1a, which has not been checked by the receiver 1b.

Display of the check-needed information 16 enables the receiver 1b to easily check the text information 5 not having been checked by the receiver 1b. This enables the receiver 1b to safely check what the speaker 1a is like such as the expression and the gesture of the speaker 1a.

Further, when the receiver 1b is concentrating on the text information 5, the text information 5 itself is arranged such that the text information 5 represents the feeling of the speaker 1a. In other words, nonverbal information regarding the speaker 1a is presented through the text information 5, which is being viewed by the receiver 1b.

These processes enable the receiver 1b to easily check both the expression of the speaker 1a and contents of speech of the speaker 1a by viewing a word (the check-needed information 16) that remains on the display screen 6b even when the receiver 1b is paying attention to the expression of the speaker 1a. Further, the receiver 1b gets to know the feeling of the speaker 1a by arrangement on the text information 5 even when the receiver 1b is paying attention to the text information 5.

Further, when a string of words (the updated text information 15) that corresponds to a result of sound recognition is displayed late, the receiver 1b may concentrate on, for example, the expression of the speaker 1a, as described above. The receiver 1b can also check a missed string of words later as the check-needed information 16 in such a case. This makes it possible to continuously perform communication properly even when there is a time lag due to the sound recognition process.

Further, the check-needed information 16 is deleted when, for example, the receiver 1b checks the check-needed information 16, as described with reference to FIG. 15. In this case, unchecked information is displayed on the display screen 6b in principle. This enables the receiver 1b to easily check unread text information 5 when the receiver 1b moves the line of sight 3 from the text information 5 such as the check-needed information 16 or the updated text information 15 and then moves the line of sight 3 to be oriented toward the text information 5 again.

When the method for displaying the pieces of check-needed information 16 in a state of being stacked above the updated text information 15, as described with reference to, for example, FIG. 13, is adopted, the pieces of check-needed information 16 can be easily checked chronologically. This makes it possible to sufficiently reduce burdens imposed on the receiver 1b.

For example, a method for displaying a string of words in a state of being fixed to the viewpoint of the receiver 1b may be used as the method for displaying the latest text information 5 (the updated text information 15). In this case, when, for example, the viewpoint of the receiver 1b is situated on the face of the speaker 1a, a word will overlap the face of the speaker 1a, and this results in difficulty in viewing both the face of the speaker 1a and a string of words. Further, when the receiver 1b moves the line of sight 3 only for a moment to check a surrounding state, a string of words may be displayed on a portion to which the receiver 1b pays attention in order to check the surrounding state, and then the string of words may become an obstacle.

On the other hand, in the present embodiment, the latest text information 5 (the updated text information 15) is displayed at a specified position on the display screen 6 (refer to A of FIG. 6), or at a position based on the position of the face of the speaker 1a (refer to B of FIG. 6). This enables the receiver 1b to easily read the text information 5 while checking, for example, the expression of the speaker 1a.

Further, a method for particularly displaying a string of speech words provided when, for example, the line of sight 3 (the viewpoint) of the receiver 1b is situated at the position of the face of the speaker 1a may be adopted as the method for displaying the check-needed information 16. Here, the line of sight 3 of the receiver 1b may frequently move between the face of the speaker 1a and a string of speech words. Thus, when the method for simply estimating the position of the viewpoint of the receiver 1b is used, it will be difficult to determine a string of words that is to be particularly displayed. Even if the string of words that is to be particularly displayed is determined, it will be difficult to continuously display all of the determined strings of words. Thus, there is a need to delete display of a string of words as appropriate.

On the other hand, in the present embodiment, the viewing state determining section 66 determines whether the receiver 1b has viewed the text information 5, using various determination conditions (refer to, for example, FIGS. 7 to 12). This makes it possible to determine, for example, whether the line of sight 3 of the receiver 1b is oriented toward the text information 5 such that the receiver 1b can read contents of the text information 5. This makes it possible to determine, with a high degree of accuracy, the text information 5 not having been checked by the receiver 1b, and thus to set the check-needed information 16 properly.

Further, for example, the line of sight 3 of the receiver 1b or the period of time for which the check-needed information 16 is displayed, is referred to, and the displayed check-needed information 16 is deleted as appropriate (refer to FIG. 15). This results in the check-needed information 16 not unnecessarily blocking the field of view of the receiver 1b. This enables the receiver 1b to easily check both what the speaker 1a is like and contents of speech of the speaker 1a.

### OTHER EMBODIMENTS

The present technology is not limited to the embodiments described above, and can achieve various other embodiments.

The example of displaying the text information 5 to the receiver 1b has been primarily described above. The present technology can also be used to control display of the text information 5 presented to the speaker 1a.

Specifically, when the receiver 1b is in a state of not having viewed the text information 5, the output controller 67 generates a report image used to inform that the receiver 1b has not viewed the text information 5. Then, the generated report image is displayed on the smart glasses 20a used by the speaker.

This process is performed in, for example, Step 108 illustrated in FIG. 5.

For example, an image on a screen similar to the display screen 6b viewed by the receiver 1b is generated as a report image and displayed on the smart glasses 20a of the speaker 1a. In other words, the speaker 1a views the display screen 6a similar to the screen of the receiver 1b.

In this case, the check-needed information 16 is displayed when, for example, the receiver 1b has not viewed the text information 5. Thus, the display of the check-needed information 16 enables the speaker 1a to check the fact that the receiver 1b has not viewed contents of speech and the contents of speech.

The speaker 1a is informed that the receiver 1b is in a state of not having viewed the text information 5, as described above. This makes it possible to induce, depending on a level of learning performed by the speaker 1a, a speech behavior of the speaker 1a that is refraining from next speech. This enables the receiver 1b to sufficiently check both what the speaker 1a is like and contents of speech of the speaker 1a.

The process of determining a viewing state of a receiver with respect to text information on the basis of line-of-sight information regarding the line of sight of the receiver has been described in the embodiment above. Without being limited thereto, the viewing state may be determined on the basis of information other than the line-of-sight information regarding the line of sight of the receiver.

For example, the viewing state may be determined according to contents of speech. Here, a process of discretionarily outputting the determination that a receiver is in a state of not having viewed a string of words (text information) of contents of speech that satisfy a specified condition, is performed.

For example, a result of sound recognition is analyzed using a technique such as natural language processing or semantic analysis, and a degree of importance of the contents of speech is determined. Further, when a string of words of which the degree of importance exceeds a certain threshold is detected, the receiver is determined to be in a state of not having viewed the string of words regardless of whether the receiver has actually viewed the string of words. It can also be said that this is a process of extracting information that is to be viewed by the receiver, according to the degree of importance of contents of speech.

This makes it possible to provide a mechanism that leads the receiver to read important contents of speech at least twice.

Further, the viewing state may be determined according to a movement of a head of a receiver. For example, the acceleration sensor (29b) included in smart glasses used by the receiver is used to detect, for example, a pose of the head of the receiver. It is determined whether the receiver is in a state of having viewed text information according to a result of the detection.

For example, when the receiver is in a state of tilting his/her head on the left or on the right (a tilt state), it is determined that the state corresponds to a head gesture for “I don't know”. When such a head gesture is detected, the receiver is determined to be in a state of not having viewed text information.

This makes it possible to display, as check-needed information, not only text information that has not been viewed by a receiver, but also, for example, text information that the receiver does not understand.

Further, the viewing state may be determined on the basis of information regarding input operated by a receiver.

For example, a button used to inform that text information has not been viewed, may be used. This button is an input apparatus that makes it possible to forcibly determine that the receiver is in a state of not having viewed text information. When the receiver presses this button, it is determined that the receiver is in a state of not having viewed text information.

This enables a receiver to purposely perform a text information selecting process such as leaving a string of words that is desired to be checked.

Further, for example, a button used to inform that text information has been viewed, may be used. This button is an input apparatus that makes it possible to forcibly determine that the receiver is in a state of having viewed text information. When the receiver presses this button, it is determined that the receiver is in a state of having viewed text information.

This enables a receiver to purposely perform a text information selecting process such as specifying a string of words that is desired to not be left (intentionally not leaving the string of words).

Moreover, any processes that make it possible to determine, for example, a viewing state of a receiver and whether a string of words is necessary, may be performed.

In the embodiment described above, it is determined whether text information has been viewed in regard to a viewing state of a receiver with respect to text information. Without being limited thereto, for example, to what extent the receiver has viewed text information may be estimated, and display of the text information may be controlled according to a result of the estimation.

For example, when a viewpoint of the receiver remains in a region of text information for a long period of time or when the viewpoint of the receiver remains in the region of the text information frequently, it is considered that the receiver has viewed the text information to a great extent. A process including estimating to what extent the receiver has viewed text information, as described above, and displaying check-needed information when, for example, the estimated extent is lesser than a threshold, is performed.

Further, for example, the method for displaying check-needed information and the method for deleting the check-needed information may be changed depending on to what extent the receiver has viewed text information. For example, a process indicated below may be performed. Check-needed information is more highlighted to be displayed for a longer period of time if the receiver has viewed text information to a lesser extent. This enables a receiver to check, with certainty, information that the receiver has missed.

The example in which text information is displayed to both a receiver and a speaker has been described in the embodiment above. Without being limited thereto, it is sufficient if a string of words of speech of (text information regarding) the speaker is displayed on at least a display apparatus used by the receiver. Thus, the speaker does not necessarily have to use a display apparatus. In this case, text information or the like that the receiver has missed is also displayed to the receiver, and a smooth communication can be performed using the text information.

The system using the smart glasses 20a and the smart glasses 20b has been described in the embodiment above. The type of display apparatus is not limited. For example, any display devices that can be applied to technologies such as augmented reality (AR), virtual reality (VR), and mixed reality (MR) may be used. Smart glasses are, for example, an eyeglass-type HMD favorably used for, for example, AR. Moreover, for example, an immersive HMD formed to cover a head of a wearing person may be used.

Further, a portable device such as a smartphone or a tablet may be used as a display apparatus. In this case, a speaker and a receiver communicate with each other using pieces of text information respectively displayed on their smartphones.

Further, for example, digital out-of-home (DOOH) advertising, or a digital signage device that provides, for example, street user-assistance services may be used. In this case, communication is performed using text information displayed on a signage device.

Furthermore, for example, a transparent display, a PC monitor, a projector, or a TV apparatus may be used as the display apparatus. For example, contents of speech of a speaker are displayed in the form of a text on a transparent display placed at, for example, a service window. Further, the display apparatus such as a PC monitor may be used when, for example, a remote video communication is performed.

The example in which a speaker and a receiver actually communicate face-to-face with each other has been primarily described in the embodiment above. Without being limited thereto, the present technology may be applied to, for example, a conversation at a remote meeting. In this case, text information obtained by converting speech of a speaker into a text using sound recognition, is displayed on, for example, PC screens respectively used by the speaker and a receiver. On the PC screen of the receiver, a viewing state of the receiver with respect to the text information is determined, and, for example, check-needed information is displayed as appropriate according to a result of the determination.

Further, the present technology is not limited to being applied to one-on-one communication between a speaker and a receiver, and can be applied to the case in which there is a participant other than the speaker and the receiver. When, for example, a receiver who is a deaf or hard-of-hearing person talks to a plurality of speakers being people with good hearing, contents of speech of each speaker are presented to the receiver in the form of text information. A viewing state of the receiver with respect to the text information is determined, and, for example, check-needed information is displayed as appropriate according to a result of the determination. Further, for example, information regarding the viewing state of the receiver and check-needed information may be presented to each speaker.

The present technology may be used for, for example, a translation conversation used to translate contents of speech of a speaker and to inform the translated contents to a receiver. In this case, sound recognition is performed on speech of the speaker, and a recognized string of words is translated. Further, text information before translation is displayed to the speaker, and text information after translation is displayed to the receiver. Also in such a case, a viewing state of the receiver with respect to text information is determined, and, for example, a translation result that the receiver has missed is displayed as check-needed information according to a result of the determination. Further, when the receiver hears sound, the fact that check-needed information is displayed may be provided using sound feedback.

Further, the present technology may be used when a speaker makes a presentation. For example, text information (a string of words of the speech itself, or a translated string of words) that indicates contents of speech provided upon presentation is displayed in the form of captions.

In this case, a viewing state of the receiver who is looking at the presentation is determined with respect to the text information, and, for example, missed text information is displayed as check-needed information. Further, when, for example, the receiver has viewed text information, the text information may be displayed by being arranged according to representation of the speaker. This makes it possible to visualize a conversation of a presenter who makes a lot of gestures and to make a presentation that is easily understood by the receiver intuitively by visualizing a conversation in a presentation in English.

Further, the receiver may present, to the speaker, text information that the receiver has not viewed. This enables the speaker to, for example, describe again what is desired to be checked by the receiver when it has not been viewed by the receiver, and enables the speaker to inform, with certainty, the receiver of what is desired to be informed.

The example in which the information processing method according to the present technology is executed by a computer of the system controller has been described above. However, the information processing method and a program according to the present technology may be executed by the computer included in the system controller and another computer that is capable of communicating with the computer included in the system controller through, for example, a network.

In other words, the information processing method and the program according to the present technology can be executed not only in a computer system that includes a single computer, but also in a computer system in which a plurality of computers operates cooperatively. Note that, in the present disclosure, the system refers to a set of components (such as apparatuses and modules (parts)) and it does not matter whether all of the components are in a single housing. Thus, a plurality of apparatuses accommodated in separate housings and connected to each other through a network, and a single apparatus in which a plurality of modules is accommodated in a single housing are both the system.

The execution of the information processing method and the program according to the present technology by the computer system includes, for example, both the case in which the process of acquiring text information regarding a speaker, the process of acquiring line-of-sight information regarding a line of sight of a receiver, the process of estimating a viewing state of the receiver with respect to the text information, and the process of controlling display related to the text information according to the viewing state are executed by a single computer; and the case in which the respective processes are executed by different computers. Further, the execution of the respective processes by a specified computer includes causing another computer to execute a portion of or all of the processes and acquiring a result of it.

In other words, the information processing method and the program according to the present technology are also applicable to a configuration of cloud computing in which a single function is shared and cooperatively processed by a plurality of apparatuses through a network.

At least two of the features of the present technology described above can also be combined. In other words, the various features described in the respective embodiments may be combined discretionarily regardless of the embodiments. Further, the various effects described above are not limitative but are merely illustrative, and other effects may be provided.

In the present disclosure, expressions such as “same”, “equal”, and “orthogonal” include, in concept, expressions such as “substantially the same”, “substantially equal”, and “substantially orthogonal”. For example, the expressions such as “same”, “equal”, and “orthogonal” also include states within specified ranges (such as a range of +/−10%), with expressions such as “exactly the same”, “exactly equal”, and “completely orthogonal” being used as references.

Note that the present technology may also take the following configurations.

(1) An information processing apparatus, including:
a first acquisition section that acquires text information obtained by converting speech of a speaker into a text using sound recognition;
a second acquisition section that acquires line-of-sight information that indicates a line of sight of a receiver who receives the speech of the speaker;
a display controller that displays the text information on at least a display apparatus that is used by the receiver; and
an estimator that estimates, on the basis of the line-of-sight information, a viewing state of the receiver with respect to the text information displayed on the display apparatus used by the receiver,
the display controller controlling display related to the text information on the basis of the viewing state.

(2) The information processing apparatus according to (1), in which
the estimator performs a determination process of determining whether the viewing state of the receiver corresponds to a state in which the receiver has viewed the text information, and
the display controller controls the display related to the text information on the basis of a determination result obtained by the determination process related to the viewing state.

(3) The information processing apparatus according to (2), in which
the estimator performs the determination process related to the viewing state on the basis of at least one of the number of times of a back-and-forth movement of the line of sight of the receiver between a face of the speaker and the text information, a duration of the back-and-forth movement, or a remaining period of time for which the line of sight of the receiver remains on the face of the speaker.

(4) The information processing apparatus according to (2), in which
the display controller displays the text information on the display apparatus used by the receiver in a state in which the text information is moving, and
the estimator performs the determination process related to the viewing state on the basis of a following period of time for which the line of sight of the receiver follows the moving text information.

(5) The information processing apparatus according to any one of (2) to (4), in which
according to a speed at which the text information is updated, the estimator changes a determination threshold used to perform the determination process related to the viewing state.

(6) The information processing apparatus according to any one of (2) to (5), in which
when the receiver is in a state of not having viewed the text information, the display controller sets, as check-needed information, the text information not having been viewed by the receiver, and causes the check-needed information to remain displayed on the display apparatus used by the receiver.

(7) The information processing apparatus according to (6), in which
the display controller displays the check-needed information in a display mode that is different from a display mode used for other text information that is displayed on the same display screen as the check-needed information.

(8) The information processing apparatus according to (6) or (7), in which
the display controller
   determines a string of words, from among strings of words included in the check-needed information, that has been read by the receiver, and
   displays, in different display modes, the string of words having been read by the receiver and a string of words that has not been read by the receiver.

(9) The information processing apparatus according to any one of (6) to (8), in which
the display controller displays the check-needed information on the display apparatus used by the receiver, such that the check-needed information is stacked above the newly displayed text information.

(10) The information processing apparatus according to any one of (6) to (8), in which
the display apparatus used by the receiver is a transmissive display apparatus, and
the display controller displays the check-needed information such that the check-needed information overlaps the face of the speaker.

(11) The information processing apparatus according to any one of (6) to (8), in which
the display apparatus used by the receiver is a transmissive display apparatus, and
the display controller displays the check-needed information on a background viewed through the displayed apparatus used by the receiver, such that the check-needed information overlaps a portion of the background that makes the display of the check-needed information noticeable.

(12) The information processing apparatus according to any one of (6) to (11), further including
an unnecessary information determining section that determines unnecessary information from among the check-needed information displayed on the display apparatus used by the receiver, in which
the display controller deletes the display of the check-needed information determined to be the unnecessary information.

(13) The information processing apparatus according to (12), in which
on the basis of the line-of-sight information regarding the line of sight of the receiver, the unnecessary information determining section determines whether the receiver has checked the check-needed information, and
the unnecessary information determining section determines, as the unnecessary information, the check-needed information determined to have been checked by the receiver.

(14) The information processing apparatus according to (12) or (13), in which
according to how frequently the receiver looks at the face of the speaker, the unnecessary information determining section changes a determination threshold used to perform a process of the determination related to the unnecessary information.

(15) The information processing apparatus according to (12), in which
the unnecessary information determining section determines, as the piece of unnecessary information, at least one of the piece of check-needed information displayed on the display apparatus used by the receiver for a period of time that exceeds a threshold, or the piece of check-needed information displayed on the display apparatus used by the receiver for a longest period of time at a timing at which the number of the pieces of check-needed information displayed on the display apparatus used by the receiver exceeds a threshold.

(16) The information processing apparatus according to any one of (2) to (15), further including
a feeling estimator that estimates feeling information that indicates a feeling of the speaker during speaking, in which
when the receiver is in a state of having viewed the text information, the display controller performs an arrangement process of arranging the text information according to the feeling information regarding the feeling of the speaker.

(17) The information processing apparatus according to (16), in which
the display controller
   decorates the text information according to the feeling information regarding the feeling of the speaker, or
   adds a visual effect to a portion situated around the text information, according to the feeling information regarding the feeling of the speaker.

(18) The information processing apparatus according to any one of (1) to (17), in which
when the receiver is in a state of not having viewed the text information, the display controller generates a report image used to inform that the receiver has not viewed the text information, and displays the generated report image on a display apparatus that is used by the speaker.

(19) An information processing method that is performed by a computer system, the information processing method including:
acquiring text information obtained by converting speech of a speaker into a text using sound recognition;
acquiring line-of-sight information that indicates a line of sight of a receiver who receives the speech of the speaker;
displaying the text information on at least a display apparatus that is used by the receiver;
estimating, on the basis of the line-of-sight information, a viewing state of the receiver with respect to the text information displayed on the display apparatus used by the receiver; and
controlling display related to the text information on the basis of the viewing state.

(20) A program that causes a computer system to perform a process including:
acquiring text information obtained by converting speech of a speaker into a text using sound recognition;
acquiring line-of-sight information that indicates a line of sight of a receiver who receives the speech of the speaker;
displaying the text information on at least a display apparatus that is used by the receiver;
estimating, on the basis of the line-of-sight information, a viewing state of the receiver with respect to the text information displayed on the display apparatus used by the receiver; and
controlling display related to the text information on the basis of the viewing state.

### REFERENCE SIGNS LIST

1a speaker
1b receiver
2 voice
3 line of sight
5 text information
15 updated text information
16, 16a, 16b check-needed information
18 overlap-target region
19 visualization object
20, 20a, 20b smart glasses
30, 30a, 30b display
50 system controller
52 storage
53 controller
60 line-of-sight detector
61 face recognition section
62 sound recognition section
63 expression analyzer
64 feeling analyzer
65 gesture recognition section
66 viewing state determining section
67 output controller
68 unnecessary information determining section
100 communication system

1. An information processing apparatus, comprising:
a first acquisition section that acquires text information obtained by converting speech of a speaker into a text using sound recognition;
a second acquisition section that acquires line-of-sight information that indicates a line of sight of a receiver who receives the speech of the speaker;
a display controller that displays the text information on at least a display apparatus that is used by the receiver; and
an estimator that estimates, on a basis of the line-of-sight information, a viewing state of the receiver with respect to the text information displayed on the display apparatus used by the receiver,
the display controller controlling display related to the text information on a basis of the viewing state.

2. The information processing apparatus according to claim 1, wherein
the estimator performs a determination process of determining whether the viewing state of the receiver corresponds to a state in which the receiver has viewed the text information, and
the display controller controls the display related to the text information on a basis of a determination result obtained by the determination process related to the viewing state.

3. The information processing apparatus according to claim 2, wherein
the estimator performs the determination process related to the viewing state on a basis of at least one of the number of times of a back-and-forth movement of the line of sight of the receiver between a face of the speaker and the text information, a duration of the back-and-forth movement, or a remaining period of time for which the line of sight of the receiver remains on the face of the speaker.

4. The information processing apparatus according to claim 2, wherein
the display controller displays the text information on the display apparatus used by the receiver in a state in which the text information is moving, and
the estimator performs the determination process related to the viewing state on a basis of a following period of time for which the line of sight of the receiver follows the moving text information.

5. The information processing apparatus according to claim 2, wherein
according to a speed at which the text information is updated, the estimator changes a determination threshold used to perform the determination process related to the viewing state.

6. The information processing apparatus according to claim 2, wherein
when the receiver is in a state of not having viewed the text information, the display controller sets, as check-needed information, the text information not having been viewed by the receiver, and causes the check-needed information to remain displayed on the display apparatus used by the receiver.

7. The information processing apparatus according to claim 6, wherein
the display controller displays the check-needed information in a display mode that is different from a display mode used for other text information that is displayed on the same display screen as the check-needed information.

8. The information processing apparatus according to claim 6, wherein
the display controller
determines a string of words, from among strings of words included in the check-needed information, that has been read by the receiver, and
displays, in different display modes, the string of words having been read by the receiver and a string of words that has not been read by the receiver.

9. The information processing apparatus according to claim 6, wherein
the display controller displays the check-needed information on the display apparatus used by the receiver, such that the check-needed information is stacked above the newly displayed text information.

10. The information processing apparatus according to claim 6, wherein
the display apparatus used by the receiver is a transmissive display apparatus, and
the display controller displays the check-needed information such that the check-needed information overlaps the face of the speaker.

11. The information processing apparatus according to claim 6, wherein
the display apparatus used by the receiver is a transmissive display apparatus, and
the display controller displays the check-needed information on a background viewed through the displayed apparatus used by the receiver, such that the check-needed information overlaps a portion of the background that makes the display of the check-needed information noticeable.

12. The information processing apparatus according to claim 6, further comprising
an unnecessary information determining section that determines unnecessary information from among the check-needed information displayed on the display apparatus used by the receiver, wherein
the display controller deletes the display of the check-needed information determined to be the unnecessary information.

13. The information processing apparatus according to claim 12, wherein
on a basis of the line-of-sight information regarding the line of sight of the receiver, the unnecessary information determining section determines whether the receiver has checked the check-needed information, and
the unnecessary information determining section determines, as the unnecessary information, the check-needed information determined to have been checked by the receiver.

14. The information processing apparatus according to claim 12, wherein
according to how frequently the receiver looks at the face of the speaker, the unnecessary information determining section changes a determination threshold used to perform a process of the determination related to the unnecessary information.

15. The information processing apparatus according to claim 12, wherein
the unnecessary information determining section determines, as the piece of unnecessary information, at least one of the piece of check-needed information displayed on the display apparatus used by the receiver for a period of time that exceeds a threshold, or the piece of check-needed information displayed on the display apparatus used by the receiver for a longest period of time at a timing at which the number of the pieces of check-needed information displayed on the display apparatus used by the receiver exceeds a threshold.

16. The information processing apparatus according to claim 2, further comprising
a feeling estimator that estimates feeling information that indicates a feeling of the speaker during speaking, wherein
when the receiver is in a state of having viewed the text information, the display controller performs an arrangement process of arranging the text information according to the feeling information regarding the feeling of the speaker.

17. The information processing apparatus according to claim 16, wherein
the display controller
decorates the text information according to the feeling information regarding the feeling of the speaker, or
adds a visual effect to a portion situated around the text information, according to the feeling information regarding the feeling of the speaker.

18. The information processing apparatus according to claim 1, wherein
when the receiver is in a state of not having viewed the text information, the display controller generates a report image used to inform that the receiver has not viewed the text information, and displays the generated report image on a display apparatus that is used by the speaker.

19. An information processing method that is performed by a computer system, the information processing method comprising:
acquiring text information obtained by converting speech of a speaker into a text using sound recognition;
acquiring line-of-sight information that indicates a line of sight of a receiver who receives the speech of the speaker;
displaying the text information on at least a display apparatus that is used by the receiver;
estimating, on a basis of the line-of-sight information, a viewing state of the receiver with respect to the text information displayed on the display apparatus used by the receiver; and
controlling display related to the text information on a basis of the viewing state.

20. A program that causes a computer system to perform a process comprising:
acquiring text information obtained by converting speech of a speaker into a text using sound recognition;
acquiring line-of-sight information that indicates a line of sight of a receiver who receives the speech of the speaker;
displaying the text information on at least a display apparatus that is used by the receiver;
estimating, on a basis of the line-of-sight information, a viewing state of the receiver with respect to the text information displayed on the display apparatus used by the receiver; and
controlling display related to the text information on a basis of the viewing state.
